SEQUENCE LISTING (5 sequences)

SEQ ID NO: 1 - protein, 34 aa, Artificial Sequence
  Synthetic Construct
  MISC_FEATURE (2)..(2): Xaa at position 2 is Aib
  MOD_RES (20)..(20): Lys at position 20 is chemically modified by conjugation of the epsilon-amino group of the Lys side chain with ([2-(2-aminoethoxy)-ethoxy]-acetyl)2-(gamma-Glu)-CO-(CH2)18CO2H
  MOD_RES (34)..(34): Gly at position 34 is amidated
HXQGTFTSDYSKYLDEKKAKEFVEWLLEGGPSSG

SEQ ID NO: 2 - protein, 34 aa, Artificial Sequence
  Synthetic Construct
  MISC_FEATURE (2)..(2): Xaa at position 2 is Aib
  MOD_RES (20)..(20): Lys at position 20 is chemically modified by conjugation of the epsilon-amino group of the Lys side chain with ([2-(2-aminoethoxy)-ethoxy]-acetyl)2-(gamma-Glu)2-CO-(CH2)18CO2H
  MOD_RES (34)..(34): Gly at position 34 is amidated
HXQGTFTSDYSKYLDEKKAKEFVEWLLSGGPSSG

SEQ ID NO: 3 - protein, 34 aa, Artificial Sequence; identical to SEQ ID NO: 1
  Synthetic Construct
  MISC_FEATURE (2)..(2): Xaa at position 2 is Aib
  MOD_RES (20)..(20): Lys at position 20 is chemically modified by conjugation of the epsilon-amino group of the Lys side chain with ([2-(2-aminoethoxy)-ethoxy]-acetyl)2-(gamma-Glu)-CO-(CH2)16CO2H
  MOD_RES (34)..(34): Gly at position 34 is amidated

SEQ ID NO: 4 - protein, 34 aa, Artificial Sequence; identical to SEQ ID NO: 2
  Synthetic Construct
  MISC_FEATURE (2)..(2): Xaa at position 2 is Aib
  MOD_RES (20)..(20): Lys at position 20 is chemically modified by conjugation of the epsilon-amino group of the Lys side chain with ([2-(2-aminoethoxy)-ethoxy]-acetyl)2-(gamma-Glu)2-CO-(CH2)16CO2H
  MOD_RES (34)..(34): Gly at position 34 is amidated

SEQ ID NO: 5 - protein, 34 aa, Artificial Sequence
  Synthetic Construct
  MISC_FEATURE (2)..(2): Xaa at position 2 is Aib
  MOD_RES (20)..(20): Lys at position 20 is chemically modified by conjugation of the epsilon-amino group of the Lys side chain with a C14-C24 fatty acid via a linker, wherein the linker is ([2-(2-aminoethoxy)-ethoxy]-acetyl)2-(gamma-Glu)t, wherein t is 1
  MISC_FEATURE (28)..(28): Xaa at position 28 is Glu or Ser
  MOD_RES (34)..(34): Gly at position 34 is optionally amidated
HXQGTFTSDYSKYLDEKKAKEFVEWLLXGGPSSG

(19) United States
(12) Patent Application Publication
(10) Pub. No.: US 2024/0299499 A1
(43) Pub. Date: Sep. 12, 2024

(54) Therapeutic Peptide Formulations

(71) Applicant: Eli Lilly and Company, Indianapolis, IN (US)

(72) Inventors: David Paul ALLEN, Carmel, IN (US); Dhara Pankaj DESAI, Fisher, IN (US); Ken Kangyi QIAN, Carmel, IN (US)

(21) Appl. No.: 18/258,462

(22) PCT Filed: Dec. 21, 2021
(86) PCT No.: PCT/US2021/064592

§ 371 Date: Jun. 20, 2023

Related U.S. Application Data
(60) Provisional application No. 63/129,157, filed on Dec. 22, 2020.

(51) Int. Cl. A61K 38/26; A61K 47/12; A61K 47/18; A61K 47/26; A61P 3/00

(52) U.S. Cl. CPC A61K 38/26; A61K 47/12; A61K 47/183; A61K 47/26; A61P 3/00

(57) ABSTRACT

Stable pharmaceutical formulations for therapeutic dual GLP-1 receptor/glucagon receptor agonists and methods of using such stable pharmaceutical formulations.

The present invention is in the field of medicine. More particularly, the present invention relates to pharmaceutical formulations comprising therapeutic peptides that are suitable for subcutaneous (“SQ”), intramuscular (“IM”), and/or intraperitoneal (“IP”) administration. Still more particularly, the present invention relates to pharmaceutical formulations of dual glucagon-like peptide (GLP-1) receptor and glucagon (Gcg) receptor agonist peptides. These pharmaceutical formulations comprising a dual GLP-1 receptor/Gcg receptor agonist are expected to be useful in treating at least type 2 diabetes, obesity, nonalcoholic fatty liver disease (NAFLD) and/or nonalcoholic steatohepatitis (NASH).

Pharmaceutical formulations of dual GLP-1/glucagon receptor agonists are needed for the treatment of patients with least type 2 diabetes, obesity, nonalcoholic fatty liver disease (NAFLD) and/or nonalcoholic steatohepatitis (NASH). Administration of such therapeutic peptides via SQ, IP and/or IM administration is both common and advantageous. Such routes of administration allow the therapeutic peptide to be delivered in a short period of time and allow patients to self-administer therapeutic peptides without visiting a medical practitioner. Certain concentrations of dual GLP-1/glucagon receptor agonist peptides are needed for pharmaceutical formulations so that the peptide can be delivered SC, IP and/or IM to the patient. These pharmaceutical formulations with a certain concentration of the dual GLP-1/glucagon receptor agonist peptide must maintain physical and chemical stability of the peptide. However, formulating therapeutic peptides into liquid pharmaceutical formulations suitable for SQ, IM and/or IP administration is both challenging and unpredictable.

The challenge and unpredictability associated with formulating therapeutic peptides into liquid pharmaceutical formulations suitable for SQ, IM and/or IP administration is due, in part, to the numerous properties a pharmaceutical formulation must possess to be therapeutically viable. Pharmaceutical formulations must provide stability to the therapeutic peptide in solution while, at the same time, maintaining the therapeutic peptide's functional characteristics essential for therapeutic efficacy. In addition, the liquid pharmaceutical formulation must also be safe for administration to, and well tolerated by, patients as well as being suitable for manufacturing and storage. U.S. Pat. No. 9,938,335 generally describes dual GLP-1/glucagon receptor agonist peptides administered by parenteral routes. The compound described in Example 2 of U.S. Pat. No. 9,938,335 has the sequence provided in SEQ ID NO: 1 (hereinafter referred to as Compound 1). Compound 1 is currently being evaluated for the treatment of patients with type 2 diabetes. Compound 1 is a synthetic peptide composed of thirty-four amino acid residues, one non-coded amino acid (aminoisobutyric acid (Aib)), a C-terminal amide, and a C20 fatty di-acid moiety covalently attached at lysine 20 in the sequence. The covalent linker comprises a gamma-glutamate and two PEG units. Therapeutically, the peptide is an oxyntomodulin-like acylated peptide with dual agonist activity of human glucagon-like peptide (GLP-1) and glucagon (Gcg). It independently binds and activates both the glucagon-like peptide receptor (GLP-1R) and the glucagon receptor (GcgR) on the surface of susceptible cells.

It has surprisingly been found that the compounds described in U.S. Pat. No. 9,938,335, particularly Compound 1, have sub-optimal solubility at lower pH values (e.g. pH 5.0 to 6.5). It also been found that the compounds described in U.S. Pat. No. 9,938,335, particularly Compound 1, have sub-optimal stability in certain formulations having pH values of 7.0-8.5. Pharmaceutical formulations comprising a dual GLP-1/glucagon receptor agonist peptide compound having the amino acid sequence of SEQ ID NO: 1, SEQ ID NO: 2, SEQ ID NO: 3 or SEQ ID NO: 4 are needed that avoid these observed problems.

The pharmaceutical formulations provided herein satisfy the aforementioned needs. More particularly, the pharmaceutical formulations provided herein are suitable for SQ, IM and/or IP administration of dual GLP-1/glucagon receptor agonist peptides while preserving the functional characteristics of the peptide essential for therapeutic efficacy.

Accordingly, there is provided a pharmaceutical formulation comprising:
(i) a compound of the following formula:

His-Xaa2-Gln-Gly-Thr-Phe-Thr-Ser-Asp-Tyr-Ser-Lys-Tyr-Leu-Asp-Glu-Lys-Ly s-Ala-Lys-Glu-Phe-Val-Glu-Trp-Leu-Leu-Xaa28-Gly-Gly-Pro-Ser-Ser-Gly
wherein
Xaa2 is Aib;
Xaa28 is Glu or Ser;
Lys at position 20 is chemically modified by conjugation of the epsilon-amino group of the Lys side chain with a C14-C24 fatty acid via a linker between the Lys at position 20 and the C14-C24 fatty acid, wherein the linker is ([2-(2-aminoethoxy)-ethoxy]-acetyl)2-(γ-Glu)t, wherein t is 1 or 2; and the C-terminal amino acid is optionally amidated (SEQ ID NO: 5), or a pharmaceutically acceptable salt thereof;
(ii) a buffer;
(iii) a tonicity agent; and
(iii) an antioxidant,
wherein the pH of the formulation is 7.8-9.0.

It was discovered in preliminary formulation studies that compounds as described herein have sub-optimal solubility at pH 5.0-6.0. These studies revealed that the compounds should be formulated at a pH of approximately 7.0 or above to have suitable solubility for SC, IM and/or IP administration. However, further formulation studies surprisingly revealed that the compounds described herein exhibited significant stability issues at pH values ranging from 7.0 to 8.5. Further studies were performed to understand the stability issues. It has surprisingly been found that there are at least two mechanisms that may cause the stability issues. First, it was considered that the compounds may be susceptible to fibrillation because of the sequence similarity with native human glucagon. Studies described herein demonstrate that the compounds suffer significant fibrillation at a pH of less than 7.8. Second, it was considered that the compounds may be susceptible to oxidation at certain amino acid residues, notably histidine (His, H) at position 1 and tryptophan (Trp, W) at position 25. Studies described herein demonstrate that the compounds are susceptible to oxidation. The compounds are formulated as described above to address these verified causes of the stability issues. Formulating the compounds in the pH range of 7.8-9.0 avoids fibrillation. The inclusion of an antioxidant significantly reduces or eliminates the aggregates derived from oxidation of the compound.

In a further embodiment of the present invention, the C14-C24 fatty acid is a saturated monoacid or a saturated diacid selected from the group consisting of myristic acid (tetradecanoic acid)(C14 monoacid), tetradecanedioic acid (C14 diacid), palmitic acid (hexadecanoic acid)(C16 monoacid), hexadecanedioic acid (C16 diacid), margaric acid (heptadecanoic acid)(C17 monoacid), heptadecanedioic acid (C17 diacid), stearic acid (octadecanoic acid)(C18 monoacid), octadecanedioic acid (C18 diacid), nonadecylic acid (nonadecanoic acid)(C19 monoacid), nonadecanedioic acid (C19 diacid), arachadic acid (eicosanoic acid)(C20 monoacid), eicosanedioic acid (C20 diacid), heneicosylic acid (heneicosanoic acid)(C21 monoacid), heneicosanedioic acid (C21 diacid), behenic acid (docosanoic acid)(C22), docosanedioic acid (C22 diacid), lignoceric acid (tetracosanoic acid)(C24 monoacid) and tetracosanedioic acid (C24 diacid).

Preferably, the C14-C24 fatty acid is octadecanedioic acid.

Alternatively preferably, the C14-C24 fatty acid is eicosanedioic acid.

In a preferred embodiment of the present invention, the C-terminal amino acid is amidated.

In a further embodiment of the present invention, the compound is selected from the group consisting of:
(a) a compound of the following formula:

His-Xaa2-Gln-Gly-Thr-Phe-Thr-Ser-Asp-Tyr-Ser-Lys-Tyr-Leu-Asp-Glu-Lys-Ly s-Ala-Lys-Glu-Phe-Val-Glu-Trp-Leu-Leu-Glu-Gly-Gly-Pro-Ser-Ser-Gly
wherein Xaa 2 is Aib;
Lys at position 20 is chemically modified by conjugation of the epsilon-amino group of the Lys side chain with ([2-(2-aminoethoxy)-ethoxy]-acetyl)2-(γ-Glu)-CO—(CH₂)₁₈CO₂H; and
the C-terminal amino acid is amidated (SEQ ID NO: 1) (Compound 1),
or a pharmaceutically acceptable salt thereof;
(b) a compound of the following formula:

His-Xaa2-Gln-Gly-Thr-Phe-Thr-Ser-Asp-Tyr-Ser-Lys-Tyr-Leu-Asp-Glu-Lys-Ly s-Ala-Lys-Glu-Phe-Val-Glu-Trp-Leu-Leu-Ser-Gly-Gly-Pro-Ser-Ser-Gly
wherein Xaa 2 is Aib;
Lys at position 20 is chemically modified by conjugation of the epsilon-amino group of the Lys side chain with ([2-(2-aminoethoxy)-ethoxy]-acetyl)2-(γ-Glu)2-CO—(CH₂)₁₈CO₂H; and
the C-terminal amino acid is amidated (SEQ ID NO: 2)(hereinafter referred to as Compound 2),
or a pharmaceutically acceptable salt thereof;
(c) a compound of the following formula:

His-Xaa2-Gln-Gly-Thr-Phe-Thr-Ser-Asp-Tyr-Ser-Lys-Tyr-Leu-Asp-Glu-Lys-Ly s-Ala-Lys-Glu-Phe-Val-Glu-Trp-Leu-Leu-Glu-Gly-Gly-Pro-Ser-Ser-Gly
wherein Xaa 2 is Aib;
Lys at position 20 is chemically modified by conjugation of the epsilon-amino group of the Lys side chain with ([2-(2-aminoethoxy)-ethoxy]-acetyl)2-(γ-Glu)-CO—(CH₂)₁₆CO₂H; and
the C-terminal amino acid is amidated (SEQ ID NO: 3)(hereinafter referred to as Compound 3),
or a pharmaceutically acceptable salt thereof; and
(d) a compound of the following formula:

His-Xaa2-Gln-Gly-Thr-Phe-Thr-Ser-Asp-Tyr-Ser-Lys-Tyr-Leu-Asp-Glu-Lys-Ly s-Ala-Lys-Glu-Phe-Val-Glu-Trp-Leu-Leu-Ser-Gly-Gly-Pro-Ser-Ser-Gly
wherein Xaa 2 is Aib;
Lys at position 20 is chemically modified by conjugation of the epsilon-amino group of the Lys side chain with ([2-(2-aminoethoxy)-ethoxy]-acetyl)2-(γ-Glu)2-CO—(CH₂)₁₆CO₂H; and
the C-terminal amino acid is amidated (SEQ ID NO: 4)(hereinafter referred to as Compound 4),
or a pharmaceutically acceptable salt thereof.

In a preferred embodiment of the present invention, the compound has the following formula:

His-Xaa2-Gln-Gly-Thr-Phe-Thr-Ser-Asp-Tyr-Ser-Lys-Tyr-Leu-Asp-Glu-Lys-Ly s-Ala-Lys-Glu-Phe-Val-Glu-Trp-Leu-Leu-Glu-Gly-Gly-Pro-Ser-Ser-Gly
wherein Xaa 2 is Aib;
Lys at position 20 is chemically modified by conjugation of the epsilon-amino group of the Lys side chain with ([2-(2-aminoethoxy)-ethoxy]-acetyl)2-(γ-Glu)-CO—(CH₂)₁₈CO₂H; and
the C-terminal amino acid is amidated (SEQ ID NO: 1)(Compound 1),
or a pharmaceutically acceptable salt thereof.

In a further embodiment of the present invention, the formulation comprises 1 mg/mL to 100 mg/mL of the compound, or a pharmaceutically acceptable salt thereof.

Preferably, the formulation comprises 5 mg/mL to 90 mg/mL of the compound, or a pharmaceutically acceptable salt thereof.

Further preferably, the formulation comprises 10 mg/mL to 80 mg/mL of the compound, or a pharmaceutically acceptable salt thereof.

Still further preferably, the formulation comprises 20 mg/mL to 70 mg/mL of the compound, or a pharmaceutically acceptable salt thereof.

Still further preferably, the formulation comprises 30 mg/mL to 60 mg/mL of the compound, or a pharmaceutically acceptable salt thereof.

Still further preferably, the formulation comprises 40 mg/mL to 50 mg/mL of the compound, or a pharmaceutically acceptable salt thereof.

Alternatively, the formulation comprises 1 mg/mL to 50 mg/mL of the compound, or a pharmaceutically acceptable salt thereof.

Further alternatively, the formulation comprises 2 mg/mL to 45 mg/mL of the compound, or a pharmaceutically acceptable salt thereof.

Still further alternatively, the formulation comprises 3 mg/mL to 40 mg/mL of the compound, or a pharmaceutically acceptable salt thereof.

Still further alternatively, the formulation comprises 4 mg/mL to 35 mg/mL of the compound, or a pharmaceutically acceptable salt thereof.

Still further alternatively, the formulation comprises 5 mg/mL to 30 mg/mL of the compound, or a pharmaceutically acceptable salt thereof.

Still further alternatively, the formulation comprises 6 mg/mL to 25 mg/mL of the compound, or a pharmaceutically acceptable salt thereof.

Still further alternatively, the formulation comprises 7 mg/mL to 20 mg/mL of the compound, or a pharmaceutically acceptable salt thereof.

Still further alternatively, the formulation comprises 8 mg/mL to 15 mg/mL of the compound, or a pharmaceutically acceptable salt thereof.

Alternatively preferably, the formulation comprises 1 mg/mL, 2 mg/mL, 3 mg/mL, 4 mg/mL, 5 mg/mL, 6 mg/mL, 7 mg/mL, 8 mg/mL, 9 mg/mL, 10 mg/mL, 11 mg/mL, 12 mg/mL, 13 mg/mL, 14 mg/mL 15 mg/mL, 16 mg/mL, 17 mg/mL, 18 mg/mL, 19 mg/mL, 20 mg/mL, 21 mg/mL, 22 mg/mL, 23 mg/mL, 24 mg/mL, 25 mg/mL, 26 mg/mL, 27 mg/mL, 28 mg/mL, 29 mg/mL, 30 mg/mL, 31 mg/mL, 32 mg/mL, 33 mg/mL, 34 mg/mL, 35 mg/mL, 36 mg/mL, 37 mg/mL, 38 mg/mL, 39 mg/mL, 40 mg/mL, 41 mg/mL, 42 mg/mL, 43 mg/mL, 44 mg/mL, 45 mg/mL, 46 mg/mL, 47 mg/mL, 48 mg/mL, 49 mg/mL, 50 mg/mL, 55 mg/mL, 60 mg/mL, 65 mg/mL, 70 mg/mL, 75 mg/mL, 80 mg/mL, 85 mg/mL, 90 mg/mL, 95 mg/mL, or 100 mg/mL of the compound, or a pharmaceutically acceptable salt thereof.

In a still further embodiment of the present invention, the buffer is selected from the group consisting of a phosphate buffer, and a tris(hydroxymethyl)aminomethane (or 2-amino-2-hydroxymethyl-propane-1,3-diol[(HOCH₂)₃CNH₂]) buffer.

In a still further embodiment of the present invention, the formulation comprises 1 mM to 20 mM of buffer.

Preferably, the formulation comprises 3 mM to 18 mM of buffer.

Further preferably, the formulation comprises 5 mM to 15 mM of buffer.

Still further preferably, the formulation comprises 8 mM to 12 mM of buffer.

Still further preferably, the formulation comprises 9 mM to 11 mM of buffer.

In a still further embodiment of the present invention, the formulation comprises 1 mM of buffer, 2 mM of buffer, 3 mM of buffer, 4 mM of buffer, 5 mM of buffer, 6 mM of buffer, 7 mM of buffer, 8 mM of buffer, 9 mM of buffer, 10 mM of buffer, 11 mM of buffer, 12 mM of buffer, 13 mM of buffer, 14 mM of buffer, 15 mM of buffer, 16 mM of buffer, 17 mM of buffer, 18 mM of buffer, 19 mM of buffer, or 20 mM of buffer.

In a preferred embodiment of the present invention, the buffer is a tris(hydroxymethyl)aminomethane (Tris) buffer.

Further preferably, the formulation comprises 1 mM of Tris buffer, 2 mM of Tris buffer, 3 mM of Tris buffer, 4 mM of Tris buffer, 5 mM of Tris buffer, 6 mM of Tris buffer, 7 mM of Tris buffer, 8 mM of Tris buffer, 9 mM of Tris buffer, 10 mM of Tris buffer, 11 mM of Tris buffer, 12 mM of Tris buffer, 13 mM of Tris buffer, 14 mM of Tris buffer, 15 mM of Tris buffer, 16 mM of Tris buffer, 17 mM of Tris buffer, 18 mM of Tris buffer, 19 mM of Tris buffer, or 20 mM of Tris buffer.

More preferably, the formulation comprises 10 mM Tris buffer.

In a still further embodiment of the present invention, the tonicity agent is selected from the group consisting of mannitol, sucrose, trehalose, glycerin, propylene glycol, sodium chloride and arginine hydrochloride.

The tonicity agent is an excipient selected to modulate the tonicity of a formulation. Tonicity in general relates to the osmotic pressure of a solution usually relative to that of human blood serum. The formulation can be hypotonic, isotonic or hypertonic. The concentration of the tonicity agent depends on the desired tonicity and the molecular weight of the particular agent selected. For instance, 25 mg/mL of glycerin would have a similar effect on the tonicity of an aqueous solution as 95 mg/mL of sucrose. Other excipients may impact on the tonicity of a formulation and the concentration of the tonicity agent is modified according to the desired outcome and the molecular weight of the agent being used.

In a still further embodiment of the present invention, the formulation comprises 5 mg/mL to 150 mg/mL of the tonicity agent.

Preferably, the formulation comprises 10 mg/mL to 120 mg/mL of the tonicity agent.

Further preferably, the formulation comprises 20 mg/mL to 100 mg/mL of the tonicity agent.

Still further preferably, the formulation comprises 30 mg/mL to 80 mg/mL of the tonicity agent.

Still further preferably, the formulation comprises 40 mg/mL to 60 mg/mL of the tonicity agent.

Still further preferably, the formulation comprises 45 mg/mL to 55 mg/mL of the tonicity agent.

In a preferred embodiment of the present invention, the tonicity agent is mannitol.

Still further preferably, the formulation comprises 10 mg/mL to 90 mg/mL of mannitol.

Still further preferably, the formulation comprises 20 mg/mL to 80 mg/mL of mannitol.

Still further preferably, the formulation comprises 30 mg/mL to 70 mg/mL of mannitol.

Still further preferably, the formulation comprises 40 mg/mL to 60 mg/mL of mannitol.

Still further preferably, the formulation comprises 45 mg/mL to 55 mg/mL of mannitol.

More preferably, the formulation comprises 50 mg/mL of mannitol.

In a still further embodiment of the present invention, the antioxidant is selected from the group consisting of radical scavengers, chelators or chain terminators.

In a still further embodiment of the present invention, the formulation comprises 0.05-10.0 mg/mL of the antioxidant.

Preferably, the formulation comprises 0.1-5.0 mg/mL of the antioxidant.

Further preferably, the formulation comprises 0.2-1.0 mg/mL of the antioxidant.

Alternatively preferably, the formulation comprises 0.05 mg/mL of the antioxidant, 0.075 mg/mL of the antioxidant, 0.1 mg/mL of the antioxidant, 0.2 mg/mL of the antioxidant, 0.3 mg/mL of the antioxidant, 0.4 mg/mL of the antioxidant, 0.5 mg/mL of the antioxidant, 0.6 mg/mL of the antioxidant, 0.7 mg/mL of the antioxidant, 0.8 mg/mL of the antioxidant, 0.9 mg/mL of the antioxidant, 1.0 mg/mL of the antioxidant, 1.1 mg/mL of the antioxidant, 1.2 mg/mL of the antioxidant, 1.3 mg/mL of the antioxidant, 1.4 mg/mL of the antioxidant, 1.5 mg/mL of the antioxidant, 1.6 mg/mL of the antioxidant, 1.7 mg/mL of the antioxidant, 1.8 mg/mL of the antioxidant, 1.9 mg/mL of the antioxidant, 2.0 mg/mL of the antioxidant, 2.5 mg/mL of the antioxidant, 3.0 mg/mL of the antioxidant, 3.5 mg/mL of the antioxidant, 4.0 mg/mL of the antioxidant, 4.5 mg/mL of the antioxidant, 5.0 mg/mL of the antioxidant, 5.5 mg/mL of the antioxidant, 6.0 mg/mL of the antioxidant, 6.5 mg/mL of the antioxidant, 7.0 mg/mL of the antioxidant, 7.5 mg/mL of the antioxidant, 8.0 mg/mL of the antioxidant, 8.5 mg/mL of the antioxidant, 9.0 mg/mL of the antioxidant, 9.5 mg/mL of the antioxidant, or 10.0 mg/mL of the antioxidant.

In a preferred embodiment of the present invention, the antioxidant is a radical scavenger.

Further preferably, the antioxidant is selected from the group consisting of EDTA, citric acid, ascorbic acid, butylated hydroxytoluene (BHT), butylated hydroxy anisole (BHA), sodium sulfite, p-amino benzoic acid, glutathione, propyl gallate, cysteine, histidine, methionine, ethanol and N-acetyl cysteine.

Still further preferably, the antioxidant is EDTA.

Still further preferably, the formulation comprises 0.05-10.0 mg/mL of EDTA.

Still further preferably, the formulation comprises 0.1-5.0 mg/mL of EDTA.

Still further preferably, the formulation comprises 0.2-1.0 mg/mL of EDTA.

Alternatively preferably, the formulation comprises 0.05 mg/mL of EDTA, 0.075 mg/mL of EDTA, 0.1 mg/mL of EDTA, 0.2 mg/mL of EDTA, 0.3 mg/mL of EDTA, 0.4 mg/mL of EDTA, 0.5 mg/mL of EDTA, 0.6 mg/mL of EDTA, 0.7 mg/mL of EDTA, 0.8 mg/mL of EDTA, 0.9 mg/mL of EDTA, 1.0 mg/mL of EDTA, 1.1 mg/mL of EDTA, 1.2 mg/mL of EDTA, 1.3 mg/mL of EDTA, 1.4 mg/mL of EDTA, 1.5 mg/mL of EDTA, 1.6 mg/mL of EDTA, 1.7 mg/mL of EDTA, 1.8 mg/mL of EDTA, 1.9 mg/mL of EDTA, 2.0 mg/mL of EDTA, 2.5 mg/mL of EDTA, 3.0 mg/mL of EDTA, 3.5 mg/mL of EDTA, 4.0 mg/mL of EDTA, 4.5 mg/mL EDTA, 5.0 mg/mL of EDTA, 5.5 mg/mL of EDTA, 6.0 mg/mL of EDTA, 6.5 mg/mL of EDTA, 7.0 mg/mL of EDTA, 7.5 mg/mL of EDTA, 8.0 mg/mL of EDTA, 8.5 mg/mL of EDTA, 9.0 mg/mL of EDTA, 9.5 mg/mL of EDTA, or 10.0 mg/mL of EDTA.

More preferably, the formulation comprises 0.5 mg/mL of EDTA.

Alternatively preferably, the antioxidant is citric acid.

Still further preferably, the formulation comprises 1-20 mM citric acid.

Still further preferably, the formulation comprises 5-15 mM citric acid.

Still further preferably, the formulation comprises 8-12 mM citric acid.

Alternatively preferably, the formulation comprises 1 mM citric acid, 1.5 mM citric acid, 2 mM citric acid, 2.5 mM citric acid, 3 mM citric acid, 3.5 mM citric acid, 4 mM citric acid, 4.5 mM citric acid, 5 mM citric acid, 5.5 mM citric acid, 6 mM citric acid, 6.5 mM citric acid, 7 mM citric acid, 7.5 mM citric acid, 8 mM citric acid, 8.5 mM citric acid, 9 mM citric acid, 9.5 mM citric acid, 10 mM citric acid, 10.5 mM citric acid, 11 mM citric acid, 11.5 mM citric acid, 12 mM citric acid, 13 mM citric acid, 13.5 mM citric acid, 14 mM citric acid, 14.5 mM citric acid, 15 mM citric acid, 15.5 mM citric acid, 16 mM citric acid, 16.5 mM citric acid, 17 mM citric acid, 17.5 mM citric acid, 18 mM citric acid, 18.5 mM citric acid, 19 mM citric acid, 19.5 mM citric acid, or 20 mM citric acid.

More preferably, the formulation comprises 10 mM citric acid.

In an alternative embodiment of the present invention, the antioxidant is ascorbic acid.

In a further alternative embodiment of the present invention, the antioxidant is, butylated hydroxytoluene (BHT).

In a still further alternative embodiment of the present invention, the antioxidant is butylated hydroxy anisole (BHA).

In a still further alternative embodiment of the present invention, the antioxidant is sodium sulfite.

In a still further alternative embodiment of the present invention, the antioxidant is p-amino benzoic acid.

In a still further alternative embodiment of the present invention, the antioxidant is glutathione.

In a still further alternative embodiment of the present invention, the antioxidant is propyl gallate.

In a still further alternative embodiment of the present invention, the antioxidant is cysteine.

In a still further alternative embodiment of the present invention, the antioxidant is histidine.

In a still further alternative embodiment of the present invention, the antioxidant is methionine.

In a still further alternative embodiment of the present invention, the antioxidant is ethanol.

In a still further alternative embodiment of the present invention, the antioxidant is N-acetyl cysteine.

In a preferred embodiment of the present invention, the pH of the formulation is 8.0-8.6.

Further preferably, the pH of the formulation is 8.0-8.3.

In a preferred embodiment of the present invention, the pharmaceutical formulation comprises:
(i) 1 mg/mL to 100 mg/mL of the compound of the following formula:

His-Xaa2-Gln-Gly-Thr-Phe-Thr-Ser-Asp-Tyr-Ser-Lys-Tyr-Leu-Asp-Glu-Lys-Lys-Ala-Lys-Glu-Phe-Val-Glu-Trp-Leu-Leu-Glu-Gly-Gly-Pro-Ser-Ser-Gly
wherein Xaa 2 is Aib;
Lys at position 20 is chemically modified by conjugation of the epsilon-amino group of the Lys side chain with ([2-(2-aminoethoxy)-ethoxy]-acetyl)2-(γ-Glu)-CO—(CH2)18CO2H; and
the C-terminal amino acid is amidated (SEQ ID NO: 1)(Compound 1)
or a pharmaceutically acceptable salt thereof;
(ii) 10 mM of Tris buffer;
(iii) 46 mg/mL of mannitol;
(iv) 0.5 mg/mL of EDTA,
wherein the pH of the formulation is 8.0-8.3.

In a still further embodiment of the present invention, there is provided a method of treating and/or preventing type 2 diabetes, obesity, nonalcoholic fatty liver disease (NAFLD) and/or nonalcoholic steatohepatitis (NASH), wherein the method comprises administering to a patient a therapeutically effective amount of a pharmaceutical formulation as described herein.

In a still further embodiment of the present invention, there is provided a pharmaceutical formulation as described herein for use in the treatment and/or prevention of type 2 diabetes, obesity, NAFLD and/or NASH.

In a still further embodiment of the present invention, there is provided the use of a pharmaceutical formulation as described herein in the manufacture of a medicament for use in the treatment of type 2 diabetes, obesity, NAFLD and/or NASH.

As used herein, the expression “pharmaceutical formulation” means a solution having at least one active pharmaceutical ingredient (API) capable of exerting a biological effect in a human, at least one inactive ingredient (e.g., buffer, excipient, surfactant, etc.) which, when combined with the API, is suitable for therapeutic administration to a human. Pharmaceutical formulations of the present disclosure are stable formulations wherein the degree of degradation, modification, aggregation, loss of biological activity and the like, of therapeutic compounds therein, is acceptably controlled and does not increase unacceptably with time.

In the context of the present invention, the API is Compound 1, or a pharmaceutically acceptable salt thereof, Compound 2, or a pharmaceutically acceptable salt thereof, Compound 3, or a pharmaceutically acceptable salt thereof, or Compound 4, or a pharmaceutically acceptable salt thereof. Compounds 1, 2, 3 and 4 and pharmaceutically acceptable salts thereof and methods of making same are described in U.S. Pat. No. 9,938,335.

As used herein, the term “pharmaceutically acceptable excipient” refers to any ingredient having no therapeutic activity and having acceptable toxicity such as buffers, solvents, tonicity agents, stabilizers, antioxidants, surfactants or polymers used in formulating pharmaceutical products. They are generally safe for administering to humans according to established governmental standards, including those promulgated by the United States Food and Drug Administration.

As used herein, the term “buffer” as used herein refers to a solution that is resistant to changes in pH. A buffer can include a weak acid and its salt, or a weak base and its salt, which assist in maintaining the stability of the pH. Examples of buffers used in pharmaceutical formulations include bicarbonate buffers, carbonate buffers, citrate buffers, histidine buffers, phosphate buffers, tartrate buffers, tris(hydroxymethyl)aminomethane (or 2-amino-2-hydroxymethyl-propane-1,3-diol[(HOCH₂)₃CNH₂]) buffers, and combinations thereof. Certain of these buffers are suitable for pharmaceutical formulations administered subcutaneously. Buffers are selected for use in a pharmaceutical formulation according to the desired pH of the formulation. For example, the pH of pharmaceutical formulations of the present invention is 7.8 to 9.0. Buffers that are suitable to achieve this pH include a bicarbonate buffer, a carbonate buffer, a phosphate buffer, a tris(hydroxymethyl)aminomethane (or 2-amino-2-hydroxymethyl-propane-1,3-diol[(HOCH2)3CNH2]) buffer, and a sodium hydroxide (NaOH) buffer. Of these, phosphate buffers and Tris buffers are preferred for use in injectable formulations. The pH of the formulation may be adjusted using physiologically appropriate acids and bases as may be required to achieve the desired pH (for instance, adjustment to the pH may be necessary as the concentration of the API in the formulation is increased or decreased).

Tris(hydroxymethyl)aminomethane or a tris(hydroxymethyl)aminomethane buffer can be referred to as “TRIS”, “Tris”, “Tris base,” “Tris buffer,” “Trisamine”, “THAM” and other names, In addition, many buffers and/or buffer systems include Tris. For example, Tris˜buffered saline (“TBS”), Tris-hydrochloride buffer (“Tris-HCl”), Tris base (pH 10.6), Tris/borate/ethylene diamine tetra-acetate (“EDTA”) buffer (“TBE”), and Tris/acetate/EDTA buffer (“TAE”). Tris base often is used with Tris-HCl to prepare Tris buffers at a desired pH.

The term “tonicity agent” as used herein refers to pharmaceutically acceptable excipients used to modulate the tonicity of a formulation. Tonicity in general relates to the osmotic pressure of a solution usually relative to that of human blood serum. The formulation can be hypotonic, isotonic or hypertonic. Suitable tonicity agents include but are not limited to salts, amino acids and sugars. Preferred tonicity agents for use in the pharmaceutical formulations of the present invention include mannitol, sucrose, trehalose, propylene glycol, glycerin, sodium chloride and arginine hydrochloride.

The term “antioxidant” refers to pharmaceutically acceptable excipients that prevent oxidation of the API. Antioxidants that are suitable for use in the pharmaceutical formulations of the present invention include chelating agents (EDTA, citric acid), reactive oxygen scavengers (ascorbic acid, butylated hydroxytoluene (BHT), butylated hydroxy anisole (BHA), sodium sulfite, p-amino benzoic acid, glutathione, propyl gallate) and chain terminators (histidine, cysteine, methionine, ethanol and N-acetyl cysteine).

Alternative buffers, tonicity agents and antioxidants that may be suitable for use in the pharmaceutical formulations of the present invention are described in Remington: The Science and Practice of Pharmacy, 23^{rd} Edition, (Editor—Adeboye Adejare).

The pharmaceutical formulations described herein can include other suitable pharmaceutically acceptable excipients such as solubilizers, emulsifiers, surfactants, preservatives, colors, viscosity regulators, and stabilizers.

As may be used herein, the terms “about” or “approximately”, when used in reference to a particular recited numerical value or range of values, means that the value may vary from the recited value by no more than 10% (e.g., +/−10%). For example, as used herein, the expression “about 100” includes 90 and 110 and all values in between (e.g., 91, 92, 93, 94, etc.).

As used interchangeably herein, “treatment” and/or “treating” and/or “treat” are intended to refer to all processes wherein there may be a total elimination, slowing or delaying, reduction in severity or frequency (e.g., of flares or episodes), interruption or stopping of the progression of disease and/or symptoms thereof, but does not require a total elimination of all disease symptoms. Treatment includes administration of a pharmaceutical formulation of the present disclosure for treatment of a disease in a human that would benefit from at least one of the above-listed processes, including: (a) inhibiting further progression of disease symptoms and effects, i.e., arresting its development; (b) relieving the disease, i.e., causing an elimination or regression of disease, disease symptoms or complications thereof, and (c) preventing or reducing the frequency of disease episodes or flares. According to specific embodiments, the pharmaceutical formulations provided herein may be used in the treatment of at least one of type II diabetes, obesity, NAFLD and NASH.

As used interchangeably herein, the term “patient,” “subject” and “individual,” refers to a human. Unless otherwise noted, the subject is further characterized as having, being at risk of developing, or experiencing symptoms of a disease that would benefit from administration of a pharmaceutical formulation disclosed herein.

As used interchangeably herein, an “effective amount” or “therapeutically effective amount” of a pharmaceutical formulation of the instant disclosure refers to an amount necessary (at dosages, frequency of administration and for periods of time for a particular means of administration) to achieve the desired therapeutic result. An effective amount of pharmaceutical formulation of the present disclosure may vary according to factors such as the disease state, age, sex, and weight of the subject and the ability of the pharmaceutical formulation of the present disclosure to elicit a desired response in the subject. An effective amount is also one in which any toxic or detrimental effects of the pharmaceutical formulation of the present disclosure are outweighed by the therapeutically beneficial effects.

The pharmaceutical formulations of the present invention may be administered to a patient via parenteral administration. Parenteral administration, as understood in the medical field, refers to the injection of a dose into the body by a sterile syringe or some other drug delivery system including an autoinjector or an infusion pump. Exemplary drug delivery systems for use with the pharmaceutical formulations of the present disclosure are described in the following references, the disclosures of which are expressly incorporated herein by reference in their entirety: U.S. Patent Publication No. 2014/0054883 to Lanigan et al., filed Mar. 7, 2013 and entitled “Infusion Pump Assembly”; U.S. Pat. No. 7,291,132 to DeRuntz et al., filed Feb. 3, 2006 and entitled “Medication Dispensing Apparatus with Triple Screw Threads for Mechanical Advantage”; U.S. Pat. No. 7,517,334 to Jacobs et al., filed Sep. 18, 2006 and entitled “Medication Dispensing Apparatus with Spring-Driven Locking Feature Enabled by Administration of Final Dose”; and U.S. Pat. No. 8,734,394 to Adams et al., filed Aug. 24, 2012 and entitled “Automatic Injection Device with Delay Mechanism Including Dual Functioning Biasing Member.” Parenteral routes include IM, SQ and IP routes of administration.

### BRIEF DESCRIPTION OF THE FIGURES

FIG. 1 illustrates the concentration of Compound 1 in solution as the pH changes from approximately 5.0 to approximately 7.0.

FIG. 2a illustrates the total aggregates of Compound 1 solution formulations as measured by size-exclusion chromatography (SEC) in formulation matrices containing 10 mM phosphate buffer, with either NaCl or glycerin as the tonicity agent.

FIG. 2b illustrates the total aggregates of Compound 1 solution formulations as measured by SEC in formulation matrices containing 10 mM tris buffer, with either NaCl or glycerin as tonicity agent.

FIG. 3a illustrates the risk of Compound 1 fibrillation as a function of pH when 2 mg/mL of Compound 1 is formulated at various pH conditions and spiked with fibrils.

FIG. 3b illustrates the risk of Compound 1 fibrillation as a function of pH when 12 mg/mL of Compound 1 is formulated at various pH conditions and spiked with fibrils.

FIG. 4 is a RP-HPLC chromatogram illustrating the effect of thermal stress of storing 2 mg/mL of Compound 1 solution formulations 40° C. for up to 4 weeks.

FIG. 5a is a RP-HPLC chromatogram of 2 mg/mL of Compound 1 drug product, formulated with 0.5 mg/mL of EDTA illustrating the effect of transition metals and H₂O₂.

FIG. 5b is a RP-HPLC chromatogram of 2 mg/mL of Compound 1 drug product, formulated without EDTA illustrating the effect of transition metals and H₂O₂.

FIG. 6a illustrates the total aggregates in Compound 1 formulations stored at 5° C. as measured by size exclusion chromatography (SEC) at months 0, 1 and 3.

FIG. 6b illustrates the total aggregates in Compound 1 formulations stored at 25° C. as measured by size exclusion chromatography (SEC) at months 0, 1 and 3.

FIG. 6c illustrates the total aggregates in Compound 1 formulations stored at 30° C. as measured by size exclusion chromatography (SEC) at months 0, 1 and 3.

### EXAMPLES

### Preparation of Compounds 1, 2, 3 and 4

### Compound 1

HXaa2QGTFTSDYSKYLDEKKAKEFVEWLLEGGPSSG
wherein Xaa2 is Aib;
K at position 20 is chemically modified through conjugation to the epsilon-amino group of the K side-chain with ([2-(2-Amino-ethoxy)-ethoxy]-acetyl)2-(γGlu)1-CO—(CH₂)₁₈—CO₂H; and
the C-terminal amino acid is amidated as a C-terminal primary amide (SEQ ID NO: 1).

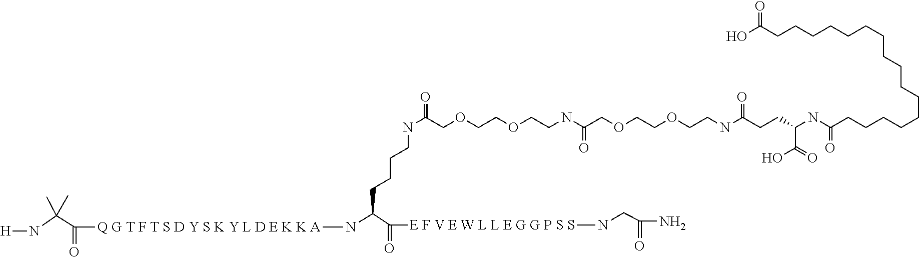

The above diagram depicts the structure of Compound 1 using the standard single letter amino acid code with the exception of residues Aib2 and K20 where the structures of these amino acids have been expanded.

Compound 1 is prepared as described in Example 2 of U.S. Pat. No. 9,938,335. An alternative method of synthesis is described in U.S. Provisional Patent Application Ser. No. 63/038,363.

### Compound 2

HXaa2QGTFTSDYSKYLDEKKAKEFVEWLLSGGPSSG
wherein Xaa2 is Aib;
K at position 20 is chemically modified through conjugation to the epsilon-amino group of the K side-chain with ([2-(2-Amino-ethoxy)-ethoxy]-acetyl)2-(γGlu)2-CO—(CH₂)₁₈—CO₂H; and
the C-terminal amino acid is amidated as a C-terminal primary amide (SEQ ID NO: 2)

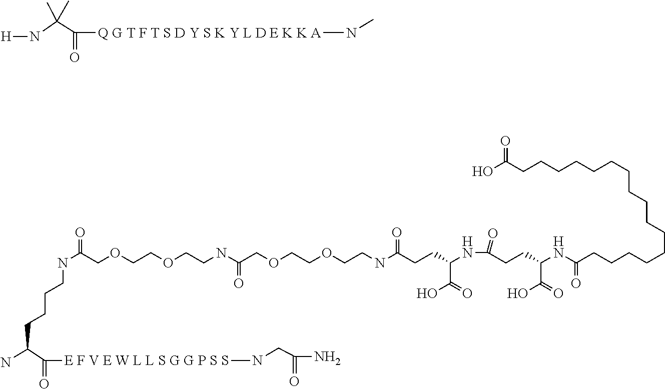

The above diagram depicts the structure Compound 2 using the standard single letter amino acid code with the exception of residues Aib2 and K20 where the structures of these amino acids have been expanded.

Compound 2 is prepared as described in Example 4 of U.S. Pat. No. 9,938,335.

### Compound 3

HXaa2QGTFTSDYSKYLDEKKAKEFVEWLLEGGPSSG
wherein Xaa2 is Aib;
K at position 20 is chemically modified through conjugation to the epsilon-amino group of the K side-chain with ([2-(2-Amino-ethoxy)-ethoxy]-acetyl)₂-(γGlu)₁-CO—(CH₂)₁₆—CO₂H; and
the C-terminal amino acid is amidated as a C-terminal primary amide (SEQ ID NO: 3).

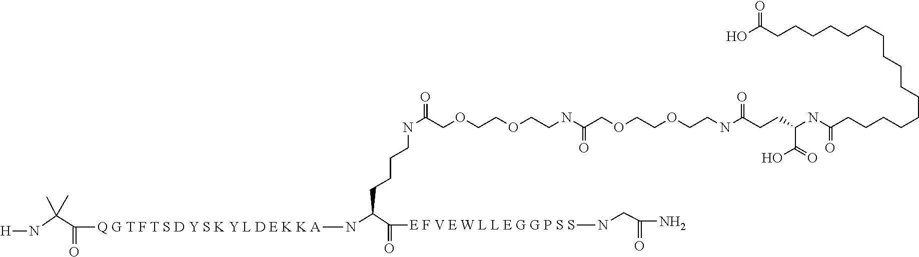

The above diagram depicts the structure of Compound 3 using the standard single letter amino acid code with the exception of residues Aib2 and K20 where the structures of these amino acids have been expanded.

Compound 3 is prepared as described in Example 1 of U.S. Pat. No. 9,938,335.

### Compound 4

HXaa2QGTFTSDYSKYLDEKKAKEFVEWLLSGGPSSG
wherein Xaa2 is Aib;
K at position 20 is chemically modified through conjugation to the epsilon-amino group of the K side-chain with ([2-(2-Amino-ethoxy)-ethoxy]-acetyl)₂-(γGlu)₂-CO—(CH₂)₁₆—CO₂H; and
the C-terminal amino acid is amidated as a C-terminal primary amide (SEQ ID NO: 4).

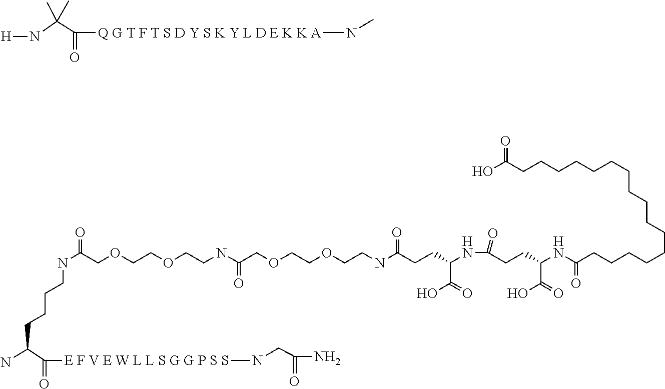

The above diagram depicts the structure of Compound 4 using the standard single letter amino acid code with the exception of residues Aib2 and K20 where the structures of these amino acids have been expanded.

Compound 4 is prepared as described in Example 3 of U.S. Pat. No. 9,938,335.

### Solubility of Compound 1

Compound 1 is being assessed in clinical trials in human patients for the treatment of type II diabetes. It is anticipated that the drug shall be administered parenterally. The solubility of Compound 1 at different pH conditions was assessed.

### Materials

The Compound 1 drug substance and excipients used in the study are detailed in Table 1. All other laboratory reagents were used as is.

TABLE 1
Materials for solubility assessment

| | | | Expiration |
|---|---|---|---|
| Ingredient | Lot No. | Supplier | date |
| Compound 1 drug | BO1704P007 | Eli Lilly/ | n/a |
| substance (QD543FA) | | Corden | |
| Tris base | C469901 | Eli Lilly | February, 2021 |
| EDTA disodium | CDBB4550V | SAFC | n/a |
| salt dihydrate | | | |
| HCl, 1N | 192001 | Fisher | April, 2021 |
| Purified water | AE29421220 | GE | July, 2022 |

### Methods

The solubility of Compound 1 was evaluated at 25° C. between pH 5.0 and pH 7.0. All solutions comprised 10 mM tris (1.21 g/L) and 0.05% EDTA (0.5 g/L). The solution pH was titrated using either 1N hydrochloric acid or concentrated tris base stock solution.

The concentration of Compound 1 was measured at 280 nm by a UV-Vis spectrophotometer (SoloVPE). UV-Vis spectrophotometer is commonly used for quantification of proteins or peptides in solution. A characteristic UV absorption spectrum around 280 nm is predominately from the aromatic amino acids such as tryptophan (Trp, W) and tyrosine (Tyr, Y). When the molar extinction coefficient of the protein or peptide is known, the Beer-Lambert law is used to accurately quantitate amount of protein or peptide by UV absorbance, assuming the molecule contains no UV-absorbing non-proteinaceous components such as bound nucleotide cofactors, heme, or iron-sulfur centers. Compound 1 has tyrosine amino acid residues at positions 10 and 13 and has a tryptophan amino acid residue at position 25 and has an extinction coefficient of 1.86 mL·mg⁻¹·cm⁻¹ (as calculated by the Pace Method). Measuring the peptide concentration at different pH conditions by this method is appropriate for Compound 1.

### Results

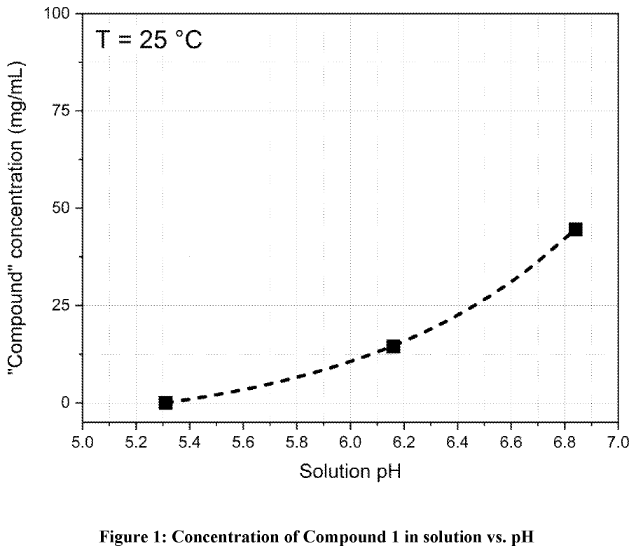

The solubility data for Compound 1 is shown in Table 2 and is illustrated in FIG. 1.

TABLE 2
Solubility data for Compound 1 at pH of 5.0-7.0 (approx.)

| | | Compound 1 |
|---|---|---|
| | | concentration in |
| Solution pH | Solvent | solution (mg/mL) |
| 5.31 | 10 mM tris (1.21 g/L) and | 0.00 |
| 6.16 | 0.05% EDTA (0.5 g/L) | 14.54 |
| 6.84 | | 44.55 |

The solubility of Compound 1 increased significantly as pH increased from 5.3 to 6.8. The study did not proceed beyond pH 6.8 due to a limited supply of Compound 1 at the time the experiment was performed. Extrapolation of the data indicates that the solubility of Compound 1 would be higher at pH values of 7.0 and above. The data indicates that Compound 1 should be formulated at pH 7.0 or higher in order to ensure adequate solubility of Compound 1 during the drug product manufacturing process and/or in the final dosage form.

### Solution Formulation Feasibility Study for Compound 1

A study was performed to assess the feasibility of formulating Compound 1 in solution. The solubility data for Compound 1 indicated that it should be formulated at pH 7.0 or higher.

### Materials

The Compound 1 API and excipients used in the study are detailed in Table 3. All other laboratory reagents were used as is.

TABLE 3
Materials for feasibility study of Compound 1

| | | | Expiration |
|---|---|---|---|
| Ingredient | Lot No. | Supplier | date |
| Compound 1 API | BO1606P002 | Eli Lilly/ | n/a |
| (QD543FA) | | Corden | |
| Phosphate buffer, 10 mM | C191775- | Eli Lilly | n/a |
| Tris buffer, 10 mM | 2017-0040 | | |
| NaOH, 1N | 164777 | Fisher | July, 2018 |
| HCl, 1N | 0000138830 | J T Baker | February, 2018 |

### Methods

The Compound 1 drug product formulations are shown in Table 4.

TABLE 4
Compound 1 drug product formulations

| Compound 1 | | Tonicity | |
|---|---|---|---|
| (mg/mL) | pH | agent | Buffer |
| 2.5 | 7.0 | NaCl, 150 mM | Phosphate buffer, 10 mM |
| 2.5 | 7.5 | | |
| 2.5 | 8.0 | | |
| 2.5 | 8.5 | | |
| 2.5 | 7.0 | Glycerin, 25 mg/mL | |
| 2.5 | 7.5 | | |
| 2.5 | 8.0 | | |
| 2.5 | 8.5 | | |
| 2.5 | 7.0 | NaCl, 150 mM | Tris buffer, 10 mM |
| 2.5 | 7.5 | | |
| 2.5 | 8.0 | | |
| 2.5 | 8.5 | | |
| 2.5 | 7.0 | Glycerin, 25 mg/mL | |
| 2.5 | 7.5 | | |
| 2.5 | 8.0 | | |
| 2.5 | 8.5 | | |

Solutions were filtered through 0.22-mm PVDF filters. In a laminar flow hood, the solutions were filled into glass vials. Vials were capped, and stored at 5° C., 25° C. and 30° C. Samples were withdrawn and submitted for testing as follows: (a) initial testing; (b) two weeks; and (c) one month. The formation of covalent aggregates was measured by size-exclusion chromatography (SEC).

### Results

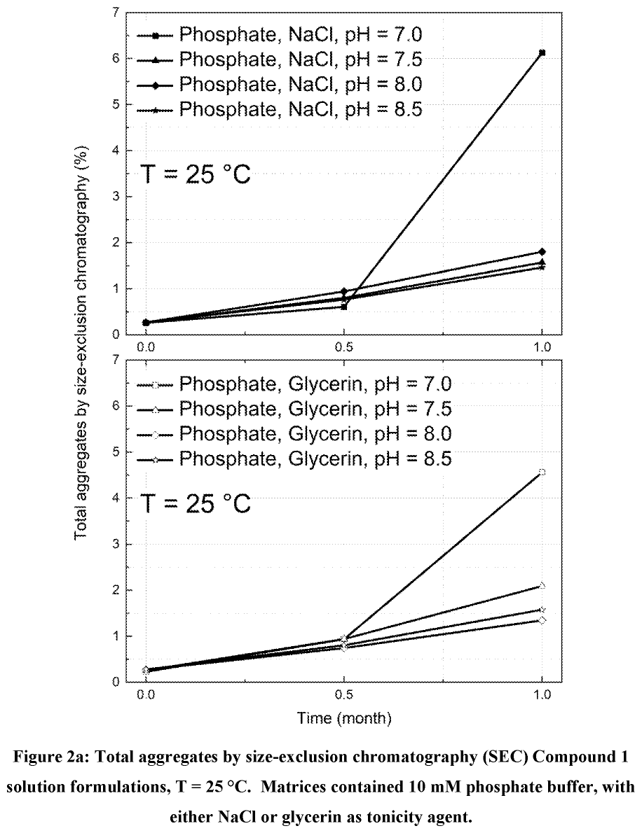

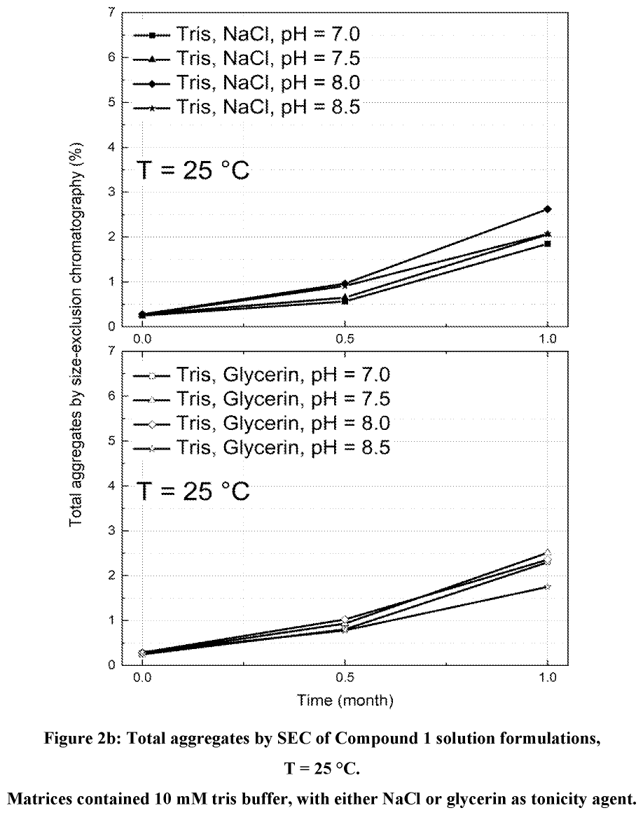

The data for the aggregate formation at 25° C. is illustrated in FIGS. 2a and 2b. Rapid aggregate formation was observed for all of the formulations. There are differences in the aggregation kinetics, depending on pH, buffer and tonicity agent. The amount of aggregates present at 25° C. after one month demonstrated that none of the formulations would be feasible as an injectable product, i.e., have a 24-month shelf life and the desired duration of in-use. Further investigations are required to understand the degradation mechanism(s) of Compound 1.

### Fibrillation Propensity and Appropriate pH Conditions for a Solution Formulation of Compound 1

Compound 1 shares some sequence similarity with native glucagon and it was considered that the compound may be susceptible to fibrillation. Fibrillation can result in loss of natural protein function, as well as potential toxic gain-of-function, such as inducing an immunogenicity response. The propensity of Compound 1 to form fibrils was evaluated to determine if it may contribute to the degradation of the compound. The risk of fibrillation of Compound 1 as a function of solution pH was evaluated.

### Materials

Solution formulations of Compound 1 at 2 mg/mL and 12 mg/mL were prepared, and the solution pH was adjusted to 7.5, 7.8, 8.0, 8.3 and 8.5, respectively. The composition of the formulation is presented in Table 5. All other laboratory reagents were used as is.

TABLE 5
Composition of solution formulation of Compound 1

| Ingredient | Supplier | Lot No. | Concentration |
|---|---|---|---|
| Compound 1^{a} | Corden/ | BO1704P007 | 2 mg/mL or 12 |
| | Eli Lilly | | mg/ml |
| Tris | Eli Lilly | C469901 | 1.21 mg/mL (10 |
| | | | mM tris buffer) |
| EDTA disodium | SAFC | CDBB4550V | 0.5 mg/mL (0.05%) |
| dihydrate | | | |
| Mannitol | Eli Lilly | C470786 | 46 mg/mL (4.6%) |
| Hydrochloric acid, 1N ^{b} | Fisher | 164777 | q.s. |
| Sodium hydroxide, 1N ^{b} | Fisher | 171239 | q.s. |
| Purified water | Hospira | 74-602-4B- | n/a - Solvent |
| | | 01 | |

^{a}The amount of Compound 1 is adjusted to account for the quantity by HPLC reported in the Certificate of Analysis.
^{b} Quantity sufficient to adjust pH

### Preparation of Compound 1 Fibril Seeds

Compound 1 fibrils were generated by incubating 500 μL of the 2 mg/mL and the 12 mg/mL solutions at pH 7.5 in a 37° C. incubator with constant end-over-end rotation for 72 hours. After 72 hours, the solution became turbid. Fibril seeds were then generated by sonicating 200 μL of the turbid solutions in a bath sonicator for various 2-minute cycles until the solution turned clear. The 2-mg/mL sample was sonicated for two 2-minute cycles, while the 12-mg/mL sample was sonicated for six 2-minute cycles.10

### Observation of Compound 1 Fibrillation by Thioflavin T (ThT) Fluorescence Assays

Compound 1 solution formulations with or without fibril seeds were prepared. For seeded samples, 5 μL of either the 2-mg/mL or 12-mg/mL seeds were added to 150 μL of corresponding drug product. For non-seeded samples, 5 μL water was added to 150 μL of Compound 1 drug product. A negative control of buffer with seed was run also.

Fibrillation of Compound 1 was observed using the thioflavin T (ThT) assays (performed as described in Schlein, M, The AAPS Journal 2017, 19, (2), 397-408). Briefly, 20 μL of each sample was added to each of three wells in a black, clear-bottom 384-well plate. ThT was added to each well, with the final ThT concentration of 4 μM per well. The plate was sealed using optical adhesive film and loaded into a SpectraMax i3x (Molecular Devices) plate reader per-incubated to 37° C. The plate was read every 15 minutes for 36 hours using an excitation wavelength of 450 nm and emission wavelength of 480 nm, with 3 second shakes between reads.

### Results: Fibrillation of Compound 1 by ThT Fluorescence Assay

Fibrils are large macromolecular self-assemblies of proteins or peptides with certain specific biophysical characteristics. Most notable is the conversion of the individual peptide backbone into a β-sheet-enriched conformation. As a result, potentially undesired physical, chemical and therapeutic risks can be raised. Experimentally, fibril formation may be visually observed as increased turbidity, precipitation, or gelation.

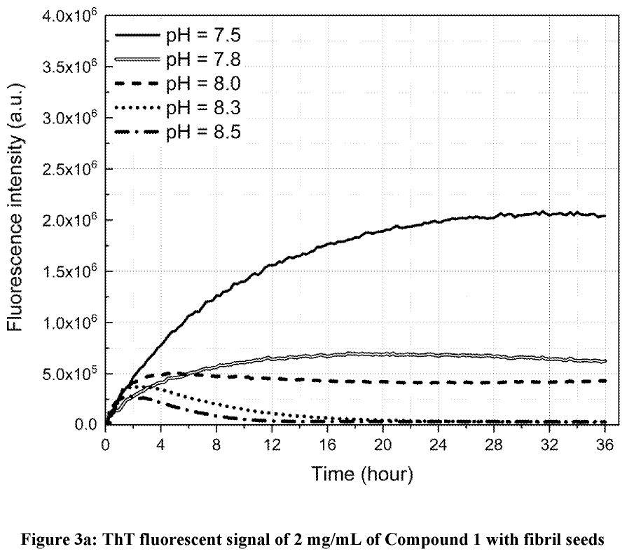

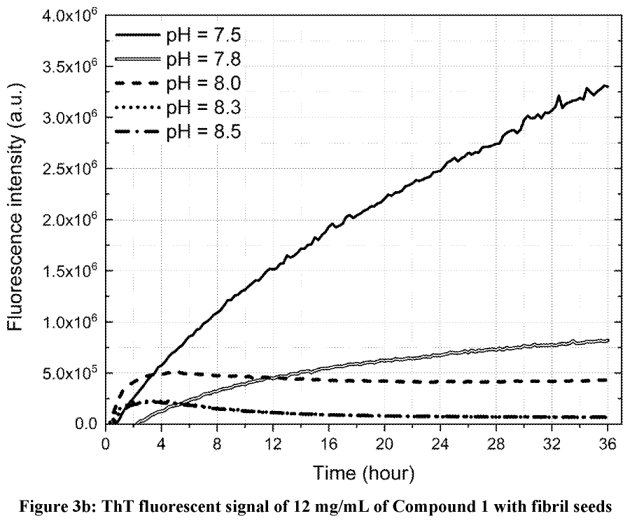

In the current study, the risk of Compound 1 fibrillation as a function of solution pH was evaluated using fluorescence spectroscopy, with thioflavin T (ThT) as the binding dye. ThT is a potent fluorescent marker of fibrils. Once selectively bound to fibril deposits, the fluorescence signal exhibits a dramatic increase in fluorescent brightness. Compound 1 was formulated at various pH conditions (7.5, 7.8, 8.0, 8.3 and 8.6), and spiked with previously-prepared fibril seeds to accelerate the fibrillation kinetics. FIGS. 3a and 3b illustrate that the increase in the ThT fluorescent signal can clearly be seen with formulations of pH at 8.0 or lower. Compound 1 fibrillation appeared to be particularly sensitive to pH. At pH below 7.8, the formation of fibrils was rapid at both concentrations (2 mg/mL and 12 mg/mL). As the pH was raised above 8.0, the fluorescent signal remained flat, indicating the absence of amyloid fibrils.

The experimental data from the ThT fluorescence assay illustrate the fibrillation risk of Compound 1 at pH≤8.0. At pH>8.0, fibrillation can be successfully prevented, even with seeding of fibrils. From the perspective of mitigating the fibrillation risk, the appropriate pH condition for the Compound 1 solution formulation is above 7.8 and preferably above 8.0. Furthermore, the study also revealed that a robust pH control during the product shelf life is critical. A suitable buffer, such as tris, with an adequate buffer strength may be utilized.

### Compound 1 Solution Formulation Degradation Study

The amino acid sequence of Compound 1 contains residues that make the peptide potentially susceptible to chemical and/or physical degradation. For example, Compound 1 has glutamine (Gln, Q) and glycine (Gly, G) at positions 3 and 4, respectively, which may be prone to deamidation. Deamidation is mainly influenced by temperature and pH. Compound 1 also has histidine (His, H) and tryptophan (Trp, W) at positions 1 and 25, respectively, which may be potential oxidation hotspots. The risk of oxidation of Compound 1 in solution was evaluated.

### Materials

Compound 1 solution formulations at 2 mg/mL and pH 8.0 were prepared. The composition of the study formulations are presented in Table 6 All other laboratory reagents were used as is.

TABLE 6
Composition of Compound 1 solution formulations

| | Compound | | Buffer matrix | | |
|---|---|---|---|---|---|
| Lot | 1 (mg/mL) | | | | Tonicity |
| No. | ^{a, b} | pH | Buffer | Stabilizer | agent |
| 1 | 2 | 8.0 | Tris, 10 | EDTA, 0.5 | Glycerin, |
| | | | mM | mg/mL | 20 mg/mL |
| 2 | 2 | 8.0 | Tris, 10 | — | Glycerin, |
| | | | mM | | 20 mg/mL |
| 3 | 2 | 8.0 | Tris, 10 | EDTA, 0.5 | Propylene |
| | | | mM | mg/mL | glycol, 15 |
| | | | | | mg/mL |
| 4 | 2 | 8.0 | Tris, 10 | — | Propylene |
| | | | mM | | glycol, 15 |
| | | | | | mg/mL |

^{a} Compound 1 API obtained from CordenPharma (Lot No.: BO1704P007)
^{b} The amount of Compound 1 is adjusted to account for the quantity by HPLC reported in the Certificate of Analysis.

### Compound 1 Degradation Stresses

Compound 1 solution formulations were subjected to various conditions, as summarized in Table 7.

TABLE 7
Composition of Compound 1 solution formulations

| Degradation | | Temperature |
|---|---|---|
| stress | Spiking condition | and duration |
| Control | None | Initial, stored |
| | | at −20° C. |
| | | for 4 weeks |
| Thermal | None | 40° C.; 4 weeks |
| Peroxide | 1 ppm of H₂O₂; 10 ppm of H₂O₂ | 40° C.; 4 weeks |
| Metal | 2 ppm of Fe³⁺; 2 ppm of Cu²⁺; | 40° C.; 4 weeks |
| | 2 ppm of Cu²⁺ + 2 ppm of Fe³⁺ + | |
| | 2 ppm of Ni²⁺ | |

### Results and Conclusions

### (a) Effect of Thermal Stress

Compound 1 solution formulations were prepared and stored at 40° C. for up to 4 weeks. The compositions of Lot 1 and Lot 3 contain 0.5 mg/ML of EDTA (Table 6), whereas the compositions of Lot 2 and Lot 4 do not comprise EDTA. EDTA is known to be an effective radical scavenger. If the oxidation of Trp is initiated by ROS, it is hypothesized that the presence of 0.5 mg/m L of EDTA may suppress the observed chemical degradation. Samples were withdrawn at appropriate times and subsequently analyzed using RP-HPLC.

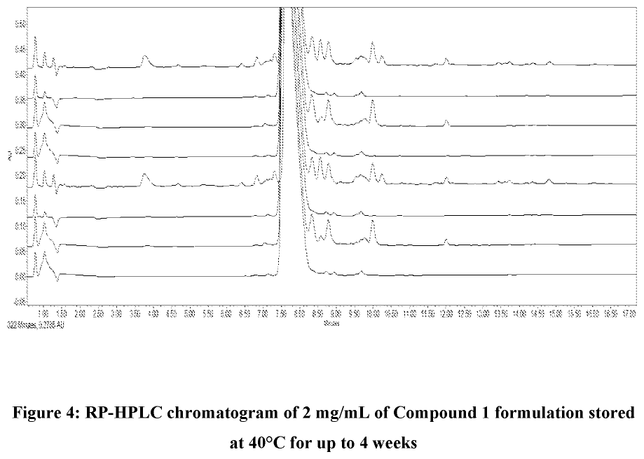

The RP-HPLC chromatograms are shown in FIG. 4. A new peak at retention time approximately 4 minutes was observed for Lot 2 and Lot 4. Based on historical data, this new peak is due to the oxidation of Trp. Similarly, another new peak was identified at retention time approximately 7 minutes, which corresponds to the double Trp oxidation. However, these new peaks are absent in the chromatograms of Lot 1 and Lot 3 at the same retention times.

(b) Effect of Transition Metals and H₂O₂

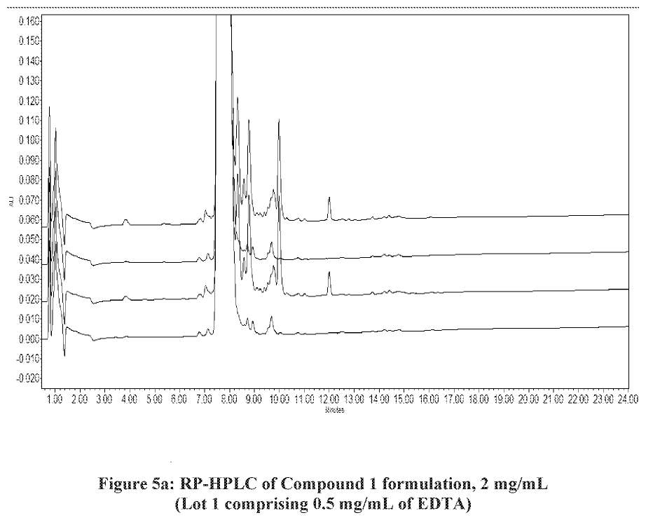

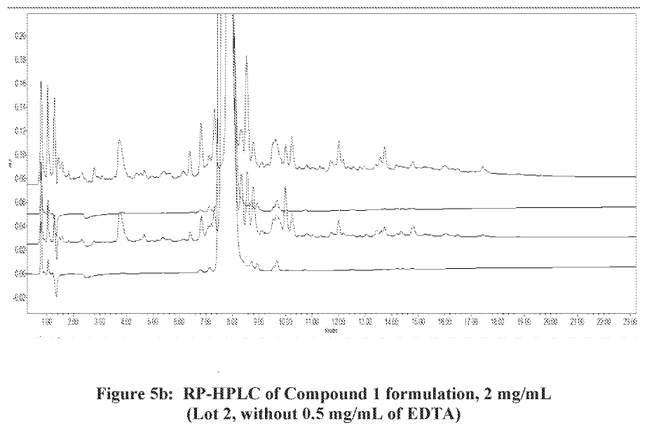

When Compound 1 formulations were spiked with 2 ppm of Fe³⁺ or 1 ppm of H₂O₂, the presence of 0.5 mg/mL EDTA suppressed Trp oxidation, i.e., absence of new peaks at retention times approximately 4 minutes and 7 minutes in the RP-HPLC chromatograms (FIG. 5a). Without EDTA, samples spiked with 2 ppm of Fe³⁺ or 1 ppm of H₂O₂ produced Trp oxidants (FIG. 5b). Compound 1 formulations spiked with Cu²⁺ or Ni²⁺ did not show the same degree of degradation.

### (c) Proposed Mitigation to Reduce Potential Oxidation in Solution

Oxidation of a protein in the solution state, with the exception of enzymatic-oxidation, is generally initiated by free radicals, i.e., reactive oxygen species (ROS). ROS can be present as a result of chemical sterilization processes, form through impurities, or with exposure to light. For example, when hydrogen peroxide (H₂O₂) is used for sterilization, residual levels of H₂O₂ can remain adsorbed on container walls and may initiate an oxidation reaction. When g-radiation is used for sterilization, ROS are generated through radiation-induced chemical processes. Transition metal ions such as iron (Fe³⁺), copper (Cu²⁺) or nickel (Ni²⁺) can be another source of ROS, and are often found in pharmaceutical preparations as impurities, either in API/drug substance or excipients. They can also be leached from equipment used to store and process protein products, such as stainless-steel vessels. Elastomer components on equipment can contain metals as well due to their curing processes. If the protein solution is exposed to light, UV light can be absorbed by aromatic amino acids, which leads to the generation of ROS.

Although all amino acid side chains are vulnerable to oxidation, radicals tend to attack preferentially a few amino acid residues, most notably, methionine (Met, M), cysteine (Cys, C), histidine (His, H) or tryptophan (Trp, W). In the amino acid sequence of Compound 1, there are His and Trp amino acids at positions 1 and 25, respectively, that make the peptide potentially prone to oxidation.

The degradation studies have shown that Compound 1 may be susceptible to oxidation. The inclusion of an antioxidant, in particular a radical scavenger (e.g., EDTA), is a plausible strategy to mitigate oxidation in the solution state.

### Compound 1 Solution Formulation Stability Study

This study evaluated the stability of Compound 1 as a solution drug product in twelve (12) formulations in pre-filled syringes at nominal, accelerated, and stressed conditions. The robustness of the formulations was assessed by varying the excipient type, pH, and addition of fibrils. The stability of Compound 1 was assessed with mannitol, sucrose, and propyl glycol as the excipients while varying the pH (8.0, 8.3, and 8.6). All formulations used in this study are listed in Table 8. The samples were stored at 5±3° C. and 25±2° C./60±5% relative humidity (RH) for up to 6 months and 30±2° C./65±5% RH for up to 2 months. The analysis schedule completed is shown in Table 9. Analytical tests that were performed at each stability time point are description by physical appearance, RP-HPLC, SEC and AEX. Test methods were executed at each stability time point.

TABLE 8
Formulations for Compound 1 Solution Formulation Feasibility Study

| | Com- | | EDTA | | | |
|---|---|---|---|---|---|---|
| | pound 1 | | Disodium | | Excipient | |
| For- | Con- | Tris | Chelating | | Con- | |
| mulation | centration | Buffer | Agent | | centration | |
| No. | (mg/mL)¹ | (mM) | (mg/mL) | Excipient | (mg/mL) | pH |
| 1 | 14.29 | 10 | 0.5 | Mannitol | 46 | 8.0 |
| 2 | | | | Mannitol | 46 | 8.3 |
| 3 | | | | Mannitol | 46 | 8.6 |
| 4 | | | | Sucrose | 90 | 8.0 |
| 5 | | | | Sucrose | 90 | 8.3 |
| 6 | | | | Sucrose | 90 | 8.6 |
| 7 | | | | Propylene | 20 | 8.0 |
| | | | | Glycol | | |
| 8 | | | | Propylene | 20 | 8.3 |
| | | | | Glycol | | |
| 9 | | | | Propylene | 20 | 8.6 |
| | | | | Glycol | | |
| 10² | | | | Mannitol | 46 | 8.3 |
| 11² | | | | Sucrose | 90 | 8.3 |
| 12² | | | | Propylene | 20 | 8.3 |
| | | | | Glycol | | |

¹The amount of Compound 1 was adjusted based upon the quantity, measured by HPLC, reported in the Certificate of Analysis. The material purity was reported as 84%. Therefore, to achieve a final concentration of 12 mg/mL, a concentration of 14.29 mg/mL was prepared.
²Formulation was spiked with fibrils seed.

TABLE 9
Testing Schedule Feasibility Study

| | Time Point (Month) | | | | | |
|---|---|---|---|---|---|---|
| Condition | T0 | 0.5 | 1 | 2 | 3 | 6 |
| 5 ± 3° C. | X | — | X | X | X | X |
| 25 ± 2° C./ | | — | X | X | X | X |
| 60 ± 5% RH | | | | | | |
| 30 ± 2° C./ | | X | X | X | — | — |
| 65 ± 5% RH | | | | | | |

### Materials

The materials used in the study are as follows:
i) Compound 1 Test Sample, Lots NB7956p7A-L, concentration of 12 mg/mL;
ii) Compound 1 Corporate Reference Standard, Lot RS1237, concentration of 0.89 mg/mL
iii) Compound 1 Fibril Seeds, Lot C241836-2018-0176-B1, concentration of 12 mg/mL
iv) Glass Prefillable Syringe Platforms, BD Neopak, C/N 47433010, Lot 4357108
v) Syringe Plungers, West Pharma, C/N 11402007, Lot D000077885
vi) EDTA Disodium Salt, Dihydrate, Sigma, C/N E1644, Lot SLBV1798
vii) Mannitol, J. T. Baker, C/N 2553-01, Lot 212595
viii) Propylene glycol, Fisher Chemical, C/N P355-1, Lot 180248
ix) Sucrose, Sigma, C/N S3929, Lot SLBV6651
x) Tris Base, Sigma, C/N T6791, Lot SLBQ2306V
xi) Aeris Peptide XB-C18, 2.6 μm, 4.6×250 mm, C/N 00G-4505-E0, Lots H18-303286, H19 051410, H19-079474, H19-084509, H19-131060, and H19-131061
xii) BioPro IEX QF, 5 μm, 4.6×100 mm, C/N QF00505-1046WP, Lot 14153 TOSOH TSKgel G2000SWXL, 5 μm, 7.8×300 mm, C/N 08540, Lots 008C-00776D and A02353-05A

### Equipment

The equipment used in this study is as follows:
i) Agilent 1100/1200 HPLC System
ii) Eisai Machinery Observation Lamp model MIH-DX
iii) Fisher Light Meter model 06-662-63
iv) HIAC Light Particle Counting System, model 9703
v) Metter Toledo SevenMulti pH meter
vi) ProteinSimple Micro-Flow Imaging Microscope, DPA 5200 with Bot 1

### Methods

### a) Description by Physical Appearance

Description by physical appearance testing at all time points was performed using the protocol entitled “Physical Appearance Testing for Client 055 Bioproduct Drug Substance and Liquid Drug Product.”

### b) RP-HPLC

The purities and protein content of the samples across all time points was determined by RP-HPLC using the protocol entitled “Identity and Purity Determination of Compound 1 by RP-HPLC.”

### c) Size Exclusion Chromatography

The aggregates of the samples across all time points was determined by size exclusion chromatography using the protocol entitled “Determination of Compound 1 Purity by Size Exclusion Chromatography.”

### d) Anion Exchange Chromatography

The charge heterogeneity profiles for the samples across all time points were determined by anion exchange chromatography using the protocol entitled “Determination of Compound 1 Charge Heterogeneity by Anion Exchange Chromatography.”

### Results

### a) Description by Physical Appearance

The physical appearance results are shown in Table 10. All formulated samples at all time points and conditions were slightly opalescent, slightly yellow liquid solutions. No particles were observed at the initial, one half month, or one month time points for each of the formulations. At the two month time point, very few particles were observed in formulations 4, 5, and 12 under the 25±2° C./60±5% RH conditions and very few particles were observed in formulations 2, 5, and 6 under the 30±2° C./65±5% RH conditions. At two months, no particles were observed in the rest of the formulations under the respective stability conditions. At the three month and six month time points, very few particles were observed in all formulations under both stability conditions.

TABLE 10
Summary of physical appearance

| | API | Composition and | Time | | Physical | | | |
|---|---|---|---|---|---|---|---|---|
| ID | (mg/mL) | Lot Number | Point | Condition | State | Color | Clarity | Particles |
| 1 | 12 | 10 mM Tris, | Initial | N/A | Liquid | Slightly | Slightly | No |
| | | 0.5 mg/mL | | | | yellow | opalescent | particles |
| | | EDTA, | 0.5M | 30 ± 2° C./ | Liquid | Slightly | Slightly | No |
| | | 46 mg/mL | | 65 ± 5% | | yellow | opalescent | particles |
| | | Mannitol, | | RH | | | | |
| | | pH 8.0, | 1M | 5 ± 3° C. | Liquid | Slightly | Slightly | No |
| | | Lot | | | | yellow | opalescent | particles |
| | | NB7956p7A | | 25 ± 2° C./ | Liquid | Slightly | Slightly | No |
| | | | | 60 ± 5% | | yellow | opalescent | particles |
| | | | | RH | | | | |
| | | | | 30 ± 2° C./ | Liquid | Slightly | Slightly | No |
| | | | | 65 ± 5% | | yellow | opalescent | particles |
| | | | | RH | | | | |
| | | | 2M | 5 ± 3° C. | Liquid | Slightly | Slightly | No |
| | | | | | | yellow | opalescent | particles |
| | | | | 25 ± 2° C./ | Liquid | Slightly | Slightly | No |
| | | | | 60 ± 5% | | yellow | opalescent | particles |
| | | | | RH | | | | |
| | | | | 30 ± 2° C./ | Liquid | Slightly | Slightly | No |
| | | | | 65 ± 5%R | | yellow | opalescent | particles |
| | | | | H | | | | |
| | | | 3M | 5 ± 3° C. | Liquid | Slightly | Slightly | Very few |
| | | | | | | yellow | opalescent | particles |
| | | | | 25 ± 2° C./ | Liquid | Slightly | Slightly | Very few |
| | | | | 60 ± 5% | | yellow | opalescent | particles |
| | | | | RH | | | | |
| | | | 6M | 5 ± 3° C. | Liquid | Slightly | Slightly | Very few |
| | | | | | | yellow | opalescent | particles |
| | | | | 25 ± 2° C./ | Liquid | Slightly | Slightly | Very few |
| | | | | 60 ± 5% | | yellow | opalescent | particles |
| | | | | RH | | | | |
| 2 | 12 | 10 mM Tris, | Initial | N/A | Liquid | Slightly | Slightly | No |
| | | 0.5 mg/mL | | | | yellow | opalescent | particles |
| | | EDTA, | 0.5M | 30 ± 2° C./ | Liquid | Slightly | Slightly | No |
| | | 46 mg/mL | | 65 ± 5% | | yellow | opalescent | particles |
| | | Mannitol, | | RH | | | | |
| | | pH 8.3, | 1M | 5 ± 3° C. | Liquid | Slightly | Slightly | No |
| | | Lot | | | | yellow | opalescent | particles |
| | | NB7956p7B | | 25 ± 2° C./ | Liquid | Slightly | Slightly | No |
| | | | | 60 ± 5% | | yellow | opalescent | particles |
| | | | | RH | | | | |
| | | | | 30 ± 2° C./ | Liquid | Slightly | Slightly | No |
| | | | | 65 ± 5% | | yellow | opalescent | particles |
| | | | | RH | | | | |
| | | | 2M | 5 ± 3° C. | Liquid | Slightly | Slightly | No |
| | | | | | | yellow | opalescent | particles |
| | | | | 25 ± 2° C./ | Liquid | Slightly | Slightly | No |
| | | | | 60 ± 5% | | yellow | opalescent | particles |
| | | | | RH | | | | |
| | | | | 30 ± 2° C./ | Liquid | Slightly | Slightly | Very few |
| | | | | 65 ± 5% | | yellow | opalescent | particles |
| | | | | RH | | | | |
| | | | 3M | 5 ± 3° C. | Liquid | Slightly | Slightly | Very few |
| | | | | | | yellow | opalescent | particles |
| | | | | 25 ± 2° C./ | Liquid | Slightly | Slightly | Very few |
| | | | | 60 ± 5% | | yellow | opalescent | particles |
| | | | | RH | | | | |
| | | | 6M | 5 ± 3° C. | Liquid | Slightly | Slightly | Very few |
| | | | | | | yellow | opalescent | particles |
| | | | | 25 ± 2° C./ | Liquid | Slightly | Slightly | Very few |
| | | | | 60 ± 5% | | yellow | opalescent | particles |
| | | | | RH | | | | |
| 3 | 12 | 10 mM Tris, | Initial | N/A | Liquid | Slightly | Slightly | No |
| | | 0.5 mg/mL | | | | yellow | opalescent | particles |
| | | EDTA, | 0.5M | 30 ± 2° C./ | Liquid | Slightly | Slightly | No |
| | | 46 mg/mL | | 65 ± 5% | | yellow | opalescent | particles |
| | | Mannitol, | | RH | | | | |
| | | pH 8.6, | 1M | 5 ± 3° C. | Liquid | Slightly | Slightly | No |
| | | Lot | | | | yellow | opalescent | particles |
| | | NB7956p7C | | 25 ± 2° C./ | Liquid | Slightly | Slightly | No |
| | | | | 60 ± 5% | | yellow | opalescent | particles |
| | | | | RH | | | | |
| | | | | 30 ± 2° C./ | Liquid | Slightly | Slightly | No |
| | | | | 65 ± 5% | | yellow | opalescent | particles |
| | | | | RH | | | | |
| | | | 2M | 5 ± 3° C. | Liquid | Slightly | Slightly | No |
| | | | | | | yellow | opalescent | particles |
| | | | | 25 ± 2° C./ | Liquid | Slightly | Slightly | No |
| | | | | 60 ± 5% | | yellow | opalescent | particles |
| | | | | RH | | | | |
| | | | | 30 ± 2° C./ | Liquid | Slightly | Slightly | No |
| | | | | 65 ± 5% | | yellow | opalescent | particles |
| | | | | RH | | | | |
| | | | 3M | 5 ± 3° C. | Liquid | Slightly | Slightly | Very few |
| | | | | | | yellow | opalescent | particles |
| | | | | 25 ± 2° C./ | Liquid | Slightly | Slightly | Very few |
| | | | | 60 ± 5% | | yellow | opalescent | particles |
| | | | | RH | | | | |
| | | | 6M | 5 ± 3° C. | Liquid | Slightly | Slightly | Very few |
| | | | | | | yellow | opalescent | particles |
| | | | | 25 ± 2° C./ | Liquid | Slightly | Slightly | Very few |
| | | | | 60 ± 5% | | yellow | opalescent | particles |
| | | | | RH | | | | |
| 4 | 12 | 10 mM Tris, | Initial | N/A | Liquid | Slightly | Slightly | No |
| | | 0.5 mg/mL | | | | yellow | opalescent | particles |
| | | EDTA, | 0.5M | 30 ± 2° C./ | Liquid | Slightly | Slightly | No |
| | | 90 mg/mL | | 65 ± 5% | | yellow | opalescent | particles |
| | | Sucrose, | | RH | | | | |
| | | pH 8.0, | 1M | 5 ± 3° C. | Liquid | Slightly | Slightly | No |
| | | Lot | | | | yellow | opalescent | particles |
| | | NB7956p7D | | 25 ± 2° C./ | Liquid | Slightly | Slightly | No |
| | | | | 60 ± 5% | | yellow | opalescent | particles |
| | | | | RH | | | | |
| | | | | 30 ± 2° C./ | Liquid | Slightly | Slightly | No |
| | | | | 65 ± 5% | | yellow | opalescent | particles |
| | | | | RH | | | | |
| | | | 2M | 5 ± 3° C. | Liquid | Slightly | Slightly | No |
| | | | | | | yellow | opalescent | particles |
| | | | | 25 ± 2° C./ | Liquid | Slightly | Slightly | Very few |
| | | | | 60 ± 5% | | yellow | opalescent | particles |
| | | | | RH | | | | |
| | | | | 30 ± 2° C./ | Liquid | Slightly | Slightly | No |
| | | | | 65 ± 5% | | yellow | opalescent | particles |
| | | | | RH | | | | |
| | | | 3M | 5 ± 3° C. | Liquid | Slightly | Slightly | Very few |
| | | | | | | yellow | opalescent | particles |
| | | | | 25 ± 2° C./ | Liquid | Slightly | Slightly | Very few |
| | | | | 60 ± 5% | | yellow | opalescent | particles |
| | | | | RH | | | | |
| | | | 6M | 5 ± 3° C. | Liquid | Slightly | Slightly | Very few |
| | | | | | | yellow | opalescent | particles |
| | | | | 25 ± 2° C./ | Liquid | Slightly | Slightly | Very few |
| | | | | 60 ± 5% | | yellow | opalescent | particles |
| | | | | RH | | | | |
| 5 | 12 | 10 mM Tris, | Initial | N/A | Liquid | Slightly | Slightly | No |
| | | 0.5 mg/mL | | | | yellow | opalescent | particles |
| | | EDTA, | 0.5M | 30 ± 2° C./ | Liquid | Slightly | Slightly | No |
| | | 90 mg/mL | | 65 ± 5% | | yellow | opalescent | particles |
| | | Sucrose, | | RH | | | | |
| | | pH 8.3, | 1M | 5 ± 3° C. | Liquid | Slightly | Slightly | No |
| | | Lot | | | | yellow | opalescent | particles |
| | | NB7956p7E | | 25 ± 2° C./ | Liquid | Slightly | Slightly | No |
| | | | | 60 ± 5% | | yellow | opalescent | particles |
| | | | | RH | | | | |
| | | | | 30 ± 2° C./ | Liquid | Slightly | Slightly | No |
| | | | | 65 ± 5% | | yellow | opalescent | particles |
| | | | | RH | | | | |
| | | | 2M | 5 ± 3° C. | Liquid | Slightly | Slightly | No |
| | | | | | | yellow | opalescent | particles |
| | | | | 25 ± 2° C./ | Liquid | Slightly | Slightly | Very few |
| | | | | 60 ± 5% | | yellow | opalescent | particles |
| | | | | RH | | | | |
| | | | | 30 ± 2° C./ | Liquid | Slightly | Slightly | Very few |
| | | | | 65 ± 5% | | yellow | opalescent | particles |
| | | | | RH | | | | |
| | | | 3M | 5 ± 3° C. | Liquid | Slightly | Slightly | Very few |
| | | | | | | yellow | opalescent | particles |
| | | | | 25 ± 2° C./ | Liquid | Slightly | Slightly | Very few |
| | | | | 60 ± 5% | | yellow | opalescent | particles |
| | | | | RH | | | | |
| | | | 6M | 5 ± 3° C. | Liquid | Slightly | Slightly | Very few |
| | | | | | | yellow | opalescent | particles |
| | | | | 25 ± 2° C./ | Liquid | Slightly | Slightly | Very few |
| | | | | 60 ± 5% | | yellow | opalescent | particles |
| | | | | RH | | | | |
| 6 | 12 | 10 mM Tris, | Initial | N/A | Liquid | Slightly | Slightly | No |
| | | 0.5 mg/mL | | | | yellow | opalescent | particles |
| | | EDTA, | 0.5M | 30 ± 2° C./ | Liquid | Slightly | Slightly | No |
| | | 90 mg/mL | | 65 ± 5% | | yellow | opalescent | particles |
| | | Sucrose, | | RH | | | | |
| | | pH 8.6, | 1M | 5 ± 3° C. | Liquid | Slightly | Slightly | No |
| | | Lot | | | | yellow | opalescent | particles |
| | | NB7956p7F | | 25 ± 2° C./ | Liquid | Slightly | Slightly | No |
| | | | | 60 ± 5% | | yellow | opalescent | particles |
| | | | | RH | | | | |
| | | | | 30 ± 2° C./ | Liquid | Slightly | Slightly | No |
| | | | | 65 ± 5% | | yellow | opalescent | particles |
| | | | | RH | | | | |
| | | | 2M | 5 ± 3° C. | Liquid | Slightly | Slightly | No |
| | | | | | | yellow | opalescent | particles |
| | | | | 25 ± 2° C./ | Liquid | Slightly | Slightly | No |
| | | | | 60 ± 5% | | yellow | opalescent | particles |
| | | | | RH | | | | |
| | | | | 30 ± 2° C./ | Liquid | Slightly | Slightly | Very few |
| | | | | 65 ± 5% | | yellow | opalescent | particles |
| | | | | RH | | | | |
| | | | 3M | 5 ± 3° C. | Liquid | Slightly | Slightly | Very few |
| | | | | | | yellow | opalescent | particles |
| | | | | 25 ± 2° C./ | Liquid | Slightly | Slightly | Very few |
| | | | | 60 ± 5% | | yellow | opalescent | particles |
| | | | | RH | | | | |
| | | | 6M | 5 ± 3° C. | Liquid | Slightly | Slightly | Very few |
| | | | | | | yellow | opalescent | particles |
| | | | | 25 ± 2° C./ | Liquid | Slightly | Slightly | Very few |
| | | | | 60 ± 5% | | yellow | opalescent | particles |
| | | | | RH | | | | |
| 7 | 12 | 10 mM Tris, | Initial | N/A | Liquid | Slightly | Slightly | No |
| | | 0.5 mg/mL | | | | yellow | opalescent | particles |
| | | EDTA, | 0.5M | 30 ± 2° C./ | Liquid | Slightly | Slightly | No |
| | | 20 mg/mL | | 65 ± 5% | | yellow | opalescent | particles |
| | | Propylene | | RH | | | | |
| | | Glycol, | 1M | 5 ± 3° C. | Liquid | Slightly | Slightly | No |
| | | pH 8.0, | | | | yellow | opalescent | particles |
| | | Lot | | 25 ± 2° C./ | Liquid | Slightly | Slightly | No |
| | | NB7956p7G | | 60 ± 5% | | yellow | opalescent | particles |
| | | | | RH | | | | |
| | | | | 30 ± 2° C./ | Liquid | Slightly | Slightly | No |
| | | | | 65 ± 5% | | yellow | opalescent | particles |
| | | | | RH | | | | |
| | | | 2M | 5 ± 3° C. | Liquid | Slightly | Slightly | No |
| | | | | | | yellow | opalescent | particles |
| | | | | 25 ± 2° C./ | Liquid | Slightly | Slightly | No |
| | | | | 60 ± 5% | | yellow | opalescent | particles |
| | | | | RH | | | | |
| | | | | 30 ± 2° C./ | Liquid | Slightly | Slightly | No |
| | | | | 65 ± 5% | | yellow | opalescent | particles |
| | | | | RH | | | | |
| | | | 3M | 5 ± 3° C. | Liquid | Slightly | Slightly | Very few |
| | | | | | | yellow | opalescent | particles |
| | | | | 25 ± 2° C./ | Liquid | Slightly | Slightly | Very few |
| | | | | 60 ± 5% | | yellow | opalescent | particles |
| | | | | RH | | | | |
| | | | 6M | 5 ± 3° C. | Liquid | Slightly | Slightly | Very few |
| | | | | | | yellow | opalescent | particles |
| | | | | 25 ± 2° C./ | Liquid | Slightly | Slightly | Very few |
| | | | | 60 ± 5% | | yellow | opalescent | particles |
| | | | | RH | | | | |
| 8 | 12 | 10 mM Tris, | Initial | N/A | Liquid | Slightly | Slightly | No |
| | | 0.5 mg/mL | | | | yellow | opalescent | particles |
| | | EDTA, | 0.5M | 30 ± 2° C./ | Liquid | Slightly | Slightly | No |
| | | 20 mg/mL | | 65 ± 5% | | yellow | opalescent | particles |
| | | Propylene | | RH | | | | |
| | | Glycol, | 1M | 5 ± 3° C. | Liquid | Slightly | Slightly | No |
| | | pH 8.3, | | | | yellow | opalescent | particles |
| | | NB7956p7H | | 25 ± 2° C./ | Liquid | Slightly | Slightly | No |
| | | | | 60 ± 5% | | yellow | opalescent | particles |
| | | | | RH | | | | |
| | | | | 30 ± 2° C./ | Liquid | Slightly | Slightly | No |
| | | | | 65 ± 5% | | yellow | opalescent | particles |
| | | | | RH | | | | |
| | | | 2M | 5 ± 3° C. | Liquid | Slightly | Slightly | No |
| | | | | | | yellow | opalescent | particles |
| | | | | 25 ± 2° C./ | Liquid | Slightly | Slightly | No |
| | | | | 60 ± 5% | | yellow | opalescent | particles |
| | | | | RH | | | | |
| | | | | 30 ± 2° C./ | Liquid | Slightly | Slightly | No |
| | | | | 65 ± 5% | | yellow | opalescent | particles |
| | | | | RH | | | | |
| | | | 3M | 5 ± 3° C. | Liquid | Slightly | Slightly | Very few |
| | | | | | | yellow | opalescent | particles |
| | | | | 25 ± 2° C./ | Liquid | Slightly | Slightly | Very few |
| | | | | 60 ± 5% | | yellow | opalescent | particles |
| | | | | RH | | | | |
| | | | 6M | 5 ± 3° C. | Liquid | Slightly | Slightly | Very few |
| | | | | | | yellow | opalescent | particles |
| | | | | 25 ± 2° C./ | Liquid | Slightly | Slightly | Very few |
| | | | | 60 ± 5% | | yellow | opalescent | particles |
| | | | | RH | | | | |
| 9 | 12 | 10 mM Tris, | Initial | N/A | Liquid | Slightly | Slightly | No |
| | | 0.5 mg/mL | | | | yellow | opalescent | particles |
| | | EDTA, | 0.5M | 30 ± 2° C./ | Liquid | Slightly | Slightly | No |
| | | 20 mg/mL | | 65 ± 5% | | yellow | opalescent | particles |
| | | Propylene | | RH | | | | |
| | | Glycol, | 1M | 5 ± 3° C. | Liquid | Slightly | Slightly | No |
| | | pH 8.6, | | | | yellow | opalescent | particles |
| | | Lot | | 25 ± 2° C./ | Liquid | Slightly | Slightly | No |
| | | NB7956p7I | | 60 ± 5% | | yellow | opalescent | particles |
| | | | | RH | | | | |
| | | | | 30 ± 2° C./ | Liquid | Slightly | Slightly | No |
| | | | | 65 ± 5% | | yellow | opalescent | particles |
| | | | | RH | | | | |
| | | | 2M | 5 ± 3° C. | Liquid | Slightly | Slightly | No |
| | | | | | | yellow | opalescent | particles |
| | | | | 25 ± 2° C./ | Liquid | Slightly | Slightly | No |
| | | | | 60 ± 5% | | yellow | opalescent | particles |
| | | | | RH | | | | |
| | | | | 30 ± 2° C./ | Liquid | Slightly | Slightly | No |
| | | | | 65 ± 5% | | yellow | opalescent | particles |
| | | | | RH | | | | |
| | | | 3M | 5 ± 3° C. | Liquid | Slightly | Slightly | Very few |
| | | | | | | yellow | opalescent | particles |
| | | | | 25 ± 2° C./ | Liquid | Slightly | Slightly | Very few |
| | | | | 60 ± 5% | | yellow | opalescent | particles |
| | | | | RH | | | | |
| | | | 6M | 5 ± 3° C. | Liquid | Slightly | Slightly | Very few |
| | | | | | | yellow | opalescent | particles |
| | | | | 25 ± 2° C./ | Liquid | Slightly | Slightly | Very few |
| | | | | 60 ± 5% | | yellow | opalescent | particles |
| | | | | RH | | | | |
| 10 | 12 | 10 mM Tris, | Initial | N/A | Liquid | Slightly | Slightly | No |
| | | 0.5 mg/mL | | | | yellow | opalescent | particles |
| | | EDTA, | 0.5M | 30 ± 2° C./ | Liquid | Slightly | Slightly | No |
| | | 46 mg/mL | | 65 ± 5% | | yellow | opalescent | particles |
| | | Mannitol, | | RH | | | | |
| | | pH 8.3 | 1M | 5 ± 3° C. | Liquid | Slightly | Slightly | No |
| | | with fibril | | | | yellow | opalescent | particles |
| | | seeds, | | 25 ± 2° C./ | Liquid | Slightly | Slightly | No |
| | | Lot | | 60 ± 5% | | yellow | opalescent | particles |
| | | NB7956p7J | | RH | | | | |
| | | | | 30 ± 2° C./ | Liquid | Slightly | Slightly | No |
| | | | | 65 ± 5% | | yellow | opalescent | particles |
| | | | | RH | | | | |
| | | | 2M | 5 ± 3° C. | Liquid | Slightly | Slightly | No |
| | | | | | | yellow | opalescent | particles |
| | | | | 25 ± 2° C./ | Liquid | Slightly | Slightly | No |
| | | | | 60 ± 5% | | yellow | opalescent | particles |
| | | | | RH | | | | |
| | | | | 30 ± 2° C./ | Liquid | Slightly | Slightly | No |
| | | | | 65 ± 5% | | yellow | opalescent | particles |
| | | | | RH | | | | |
| | | | 3M | 5 ± 3° C. | Liquid | Slightly | Slightly | Very few |
| | | | | | | yellow | opalescent | particles |
| | | | | 25 ± 2° C./ | Liquid | Slightly | Slightly | Very few |
| | | | | 60 ± 5% | | yellow | opalescent | particles |
| | | | | RH | | | | |
| | | | 6M | 5 ± 3° C. | Liquid | Slightly | Slightly | Very few |
| | | | | | | yellow | opalescent | particles |
| | | | | 25 ± 2° C./ | Liquid | Slightly | Slightly | Very few |
| | | | | 60 ± 5% | | yellow | opalescent | particles |
| | | | | RH | | | | |
| 11 | 12 | 10 mM Tris, | Initial | N/A | Liquid | Slightly | Slightly | No |
| | | 0.5 mg/mL | | | | yellow | opalescent | particles |
| | | EDTA, | 0.5M | 30 ± 2° C./ | Liquid | Slightly | Slightly | No |
| | | 90 mg/mL | | 65 ± 5% | | yellow | opalescent | particles |
| | | Sucrose, | | RH | | | | |
| | | pH 8.3 | 1M | 5 ± 3° C. | Liquid | Slightly | Slightly | No |
| | | with fibril | | | | yellow | opalescent | particles |
| | | seeds, | | 25 ± 2° C./ | Liquid | Slightly | Slightly | No |
| | | Lot | | 60 ± 5% | | yellow | opalescent | particles |
| | | NB7956p7K | | RH | | | | |
| | | | | 30 ± 2° C./ | Liquid | Slightly | Slightly | No |
| | | | | 65 ± 5% | | yellow | opalescent | particles |
| | | | | RH | | | | |
| | | | 2M | 5 ± 3° C. | Liquid | Slightly | Slightly | No |
| | | | | | | yellow | opalescent | particles |
| | | | | 25 ± 2° C./ | Liquid | Slightly | Slightly | No |
| | | | | 60 ± 5% | | yellow | opalescent | particles |
| | | | | RH | | | | |
| | | | | 30 ± 2° C./ | Liquid | Slightly | Slightly | No |
| | | | | 65 ± 5% | | yellow | opalescent | particles |
| | | | | RH | | | | |
| | | | 3M | 5 ± 3° C. | Liquid | Slightly | Slightly | Very few |
| | | | | | | yellow | opalescent | particles |
| | | | | 25 ± 2° C./ | Liquid | Slightly | Slightly | Very few |
| | | | | 60 ± 5% | | yellow | opalescent | particles |
| | | | | RH | | | | |
| | | | 6M | 5 ± 3° C. | Liquid | Slightly | Slightly | Very few |
| | | | | | | yellow | opalescent | particles |
| | | | | 25 ± 2° C./ | Liquid | Slightly | Slightly | Very few |
| | | | | 60 ± 5% | | yellow | opalescent | particles |
| | | | | RH | | | | |
| 12 | 12 | 10 mM Tris, | Initial | N/A | Liquid | Slightly | Slightly | No |
| | | 0.5 mg/mL | | | | yellow | opalescent | particles |
| | | EDTA, | 0.5M | 30 ± 2° C./ | Liquid | Slightly | Slightly | No |
| | | 20 mg/mL | | 65 ± 5% | | yellow | opalescent | particles |
| | | Propylene | | RH | | | | |
| | | Glycol, | 1M | 5 ± 3° C. | Liquid | Slightly | Slightly | No |
| | | pH 8.3 | | | | yellow | opalescent | particles |
| | | with fibril | | 25 ± 2° C./ | Liquid | Slightly | Slightly | No |
| | | seeds, | | 60 ± 5% | | yellow | opalescent | particles |
| | | Lot | | RH | | | | |
| | | NB7956p7L | | 30 ± 2° C./ | Liquid | Slightly | Slightly | No |
| | | | | 65 ± 5% | | yellow | opalescent | particles |
| | | | | RH | | | | |
| | | | 2M | 5 ± 3° C. | Liquid | Slightly | Slightly | No |
| | | | | | | yellow | opalescent | particles |
| | | | | 25 ± 2° C./ | Liquid | Slightly | Slightly | Very few |
| | | | | 60 ± 5% | | yellow | opalescent | particles |
| | | | | RH | | | | |
| | | | | 30 ± 2° C./ | Liquid | Slightly | Slightly | No |
| | | | | 65 ± 5% | | yellow | opalescent | particles |
| | | | | RH | | | | |
| | | | 3M | 5 ± 3° C. | Liquid | Slightly | Slightly | Very few |
| | | | | | | yellow | opalescent | particles |
| | | | | 25 ± 2° C./ | Liquid | Slightly | Slightly | Very few |
| | | | | 60 ± 5% | | yellow | opalescent | particles |
| | | | | RH | | | | |
| | | | 6M | 5 ± 3° C. | Liquid | Slightly | Slightly | Very few |
| | | | | | | yellow | opalescent | particles |
| | | | | 25 ± 2° C./ | Liquid | Slightly | Slightly | Very few |
| | | | | 60 ± 5% | | yellow | opalescent | particles |
| | | | | RH | | | | |

### b) Reversed-Phase High Performance Liquid Chromatography

Results for the analysis of purity by RP-HPLC are shown in Table 11. Across the set of formulations, the initial average main peak purity percentage ranged from 97.3-97.500 and the average percentage of Total Related Substances (TRS) ranged from 2.5-2.7%. Across the set of formulations at the 6M/5±3° C. time point and condition, the average main peak purity percentage ranged from 96.4-97.200 and the average percentage of TRS ranged from 2.8-3.6%. Across the set of formulations at the 6M/25±2° C./60±50% RH time point and condition, the average main peak purity percentage ranged from 89.4-93.1% and the average percentage of TRS ranged from 6.9-10.6%. Across the set of formulations at the 2M/30±2° C./65±5% RH time point and conditions, the average main peak purity percentage ranged from 92.0-94.3% and the average percentage of TRS ranged from 5.7-8.0%. For each formulation, similar patterns of decline in percentage of main peak and increase in the percentage of TRS were observed across the accelerated stability conditions. A marginal decrease in the main peak purity at the 5±3° C. condition up to 6 months in comparison to the initial value was observed. The decreased percent main peak purity was dependent on the pH value, for which, higher pH values demonstrated the most change.

The results for protein content were assessed at all time points and conditions by RP-HPLC (Table 11). Each of the twelve (12) unique formulations were prepared at a single concentration level of ˜12 mg/Ml. The protein content ranged from 9.8-13.7 mg/mL across the study. Consequently, the percent label claim ranged from 84-111% when comparing the protein concentration to the initial staged timepoint across the study. The decreased protein concentration was observed at the accelerated six-month time point condition. More specifically, the decreased content was dependent on the pH value, for which, higher pH values demonstrated the most change.

TABLE 11
Summary of RP-HPLC Results

| | | | | | | | Actual | Avg. | |
|---|---|---|---|---|---|---|---|---|---|
| | | | | | Avg. | Nominal | Avg. | Main | |
| | | | | | Qty. | Avg % | % | Peak | Avg. |
| | API | Com- | Time | | API | Label | Label | Purity | TRS |
| ID | (mg/mL) | position | Point | Condition | (mg/mL) | Claim | Claim | % | % |
| 1 | 12 | 10 mM Tris, | Initial | N/A | 11.1 | 92.5 | 97.7 | 97.4 | 2.6 |
| | | 0.5 mg/mL | 0.5M | 30 ± 2° C./ | 10.9 | 90.6 | 95.7 | 96.7 | 3.3 |
| | | EDTA, | | 65 ± 5% RH | | | | | |
| | | 46 mg/mL | 1M | 5 ± 3° C. | 10.9 | 91.0 | 96.1 | 97.4 | 2.6 |
| | | Mannitol, | | 25 ± 2° C./ | 10.8 | 90.0 | 95.1 | 96.2 | 3.8 |
| | | pH 8.0 | | 60 ± 5% RH | | | | | |
| | | | | 30 ± 2° C./ | 10.8 | 90.0 | 95.1 | 95.2 | 4.8 |
| | | | | 65 ± 5% RH | | | | | |
| | | | 2M | 5 ± 3° C. | 12.9 | 107.8 | 113.8 | 97.0 | 3.0 |
| | | | | 25 ± 2° C./ | 11.4 | 95.2 | 100.6 | 95.5 | 4.5 |
| | | | | 60 ± 5% RH | | | | | |
| | | | | 30 ± 2° C./ | 10.9 | 91.2 | 96.4 | 94.3 | 5.7 |
| | | | | 65 ± 5% RH | | | | | |
| | | | 3M | 5 ± 3° C. | 10.8 | 90.2 | 95.3 | 97.5 | 2.5 |
| | | | | 25 ± 2° C./ | 10.4 | 86.6 | 91.5 | 95.1 | 4.9 |
| | | | | 60 ± 5% RH | | | | | |
| | | | 6M | 5 ± 3° C. | 11.0 | 92.0 | 97.2 | 97.2 | 2.8 |
| | | | | 25 ± 2° C./ | 10.4 | 86.3 | 91.2 | 93.1 | 6.9 |
| | | | | 60 ± 5% RH | | | | | |
| 2 | 12 | 10 mM Tris, | Initial | N/A | 11.1 | 92.5 | 99.7 | 97.4 | 2.6 |
| | | 0.5 mg/mL | 0.5M | 30 ± 2° C./ | 11.0 | 91.4 | 98.5 | 96.6 | 3.4 |
| | | EDTA, | | 65 ± 5% RH | | | | | |
| | | 46 mg/mL | 1M | 5 ± 3° C. | 11.1 | 92.6 | 99.8 | 97.4 | 2.6 |
| | | Mannitol, | | 25 ± 2° C./ | 11.4 | 95.0 | 102.4 | 95.9 | 4.1 |
| | | pH 8.3 | | 60 ± 5% RH | | | | | |
| | | | | 30 ± 2° C./ | 10.7 | 89.5 | 96.5 | 95.1 | 4.9 |
| | | | | 65 ± 5% RH | | | | | |
| | | | 2M | 5 ± 3° C. | 12.0 | 99.6 | 107.4 | 97.2 | 2.8 |
| | | | | 25 ± 2° C./ | 11.7 | 97.9 | 105.5 | 95.2 | 4.8 |
| | | | | 60 ± 5% RH | | | | | |
| | | | | 30 ± 2° C./ | 11.1 | 92.2 | 99.4 | 93.3 | 6.7 |
| | | | | 65 ± 5% RH | | | | | |
| | | | 3M | 5 ± 3° C. | 10.6 | 88.0 | 94.9 | 97.4 | 2.6 |
| | | | | 25 ± 2° C./ | 10.2 | 85.4 | 92.0 | 94.4 | 5.6 |
| | | | | 60 ± 5% RH | | | | | |
| | | | 6M | 5 ± 3° C. | 11.1 | 92.9 | 100.1 | 97.2 | 2.8 |
| | | | | 25 ± 2° C./ | 10.2 | 84.8 | 91.4 | 92.1 | 7.9 |
| | | | | 60 ± 5% RH | | | | | |
| 3 | 12 | 10 mM Tris, | Initial | N/A | 11.3 | 93.8 | 102.5 | 97.4 | 2.6 |
| | | 0.5 mg/mL | 0.5M | 30 ± 2° C./ | 10.9 | 91.1 | 99.6 | 96.1 | 3.9 |
| | | EDTA, | | 65 ± 5% RH | | | | | |
| | | 46 mg/mL | 1M | 5 ± 3° C. | 11.3 | 94.4 | 103.2 | 97.2 | 2.8 |
| | | Mannitol, | | 25 ± 2° C./ | 10.9 | 90.5 | 99.0 | 95.8 | 4.2 |
| | | pH 8.6 | | 60 ± 5% RH | | | | | |
| | | | | 30 ± 2° C./ | 10.9 | 90.7 | 99.2 | 94.4 | 5.6 |
| | | | | 65 ± 5% RH | | | | | |
| | | | 2M | 5 ± 3° C. | 10.6 | 88.3 | 96.6 | 96.7 | 3.3 |
| | | | | 25 ± 2° C./ | 11.6 | 96.5 | 105.5 | 94.6 | 5.4 |
| | | | | 60 ± 5% RH | | | | | |
| | | | | 30 ± 2° C./ | 11.0 | 91.7 | 100.3 | 92.7 | 7.3 |
| | | | | 65 ± 5% RH | | | | | |
| | | | 3M | 5 ± 3° C. | 10.6 | 88.2 | 96.5 | 97.4 | 2.6 |
| | | | | 25 ± 2° C./ | 10.3 | 85.5 | 93.5 | 93.2 | 6.8 |
| | | | | 60 ± 5% RH | | | | | |
| | | | 6M | 5 ± 3° C. | 10.7 | 89.2 | 97.5 | 96.7 | 3.3 |
| | | | | 25 ± 2° C./ | 9.8 | 81.3 | 88.9 | 89.7 | 10.3 |
| | | | | 60 ± 5% RH | | | | | |
| 4 | 12 | 10 mM Tris, | Initial | N/A | 12.3 | 102.7 | 99.4 | 97.4 | 2.6 |
| | | 0.5 mg/mL | 0.5M | 30 ± 2° C./ | 11.9 | 99.1 | 95.9 | 96.7 | 3.3 |
| | | EDTA, | | 65 ± 5% RH | | | | | |
| | | 90 mg/mL | 1M | 5 ± 3° C. | 11.8 | 98.5 | 95.3 | 97.3 | 2.7 |
| | | Sucrose, | | 25 ± 2° C./ | 12.0 | 100.0 | 96.8 | 96.0 | 4.0 |
| | | pH 8.0 | Point | 60 ± 5% RH | | | | | |
| | | | | 30 ± 2° C./ | 11.6 | 96.8 | 93.7 | 95.2 | 4.8 |
| | | | | 65 ± 5% RH | | | | | |
| | | | 2M | 5 ± 3° C. | 13.7 | 114.1 | 110.5 | 97.1 | 2.9 |
| | | | | 25 ± 2° C./ | 12.6 | 104.7 | 101.4 | 95.3 | 4.7 |
| | | | | 60 ± 5% RH | | | | | |
| | | | | 30 ± 2° C./ | 12.3 | 102.4 | 99.1 | 93.7 | 6.3 |
| | | | | 65 ± 5% RH | | | | | |
| | | | 3M | 5 ± 3° C. | 11.3 | 94.4 | 91.4 | 97.4 | 2.6 |
| | | | | 25 ± 2° C./ | 11.4 | 94.9 | 91.8 | 94.1 | 5.9 |
| | | | | 60 ± 5% RH | | | | | |
| | | | 6M | 5 ± 3° C. | 11.5 | 95.9 | 92.9 | 96.7 | 3.3 |
| | | | | 25 ± 2° C./ | 10.7 | 88.8 | 85.9 | 92.2 | 7.8 |
| | | | | 60 ± 5% RH | | | | | |
| 5 | 12 | 10 mM Tris, | Initial | N/A | 11.9 | 99.4 | 94.9 | 97.3 | 2.7 |
| | | 0.5 mg/mL | 0.5M | 30 ± 2° C./ | 11.9 | 99.2 | 94.7 | 96.4 | 3.6 |
| | | EDTA, | | 65 ± 5% RH | | | | | |
| | | 90 mg/mL | 1M | 5 ± 3° C. | 11.6 | 96.7 | 92.3 | 97.3 | 2.7 |
| | | Sucrose, | | 25 ± 2° C./ | 12.1 | 100.9 | 96.3 | 95.7 | 4.3 |
| | | pH 8.3 | | 60 ± 5% RH | | | | | |
| | | | | 30 ± 2° C./ | 11.4 | 95.2 | 90.9 | 94.7 | 5.3 |
| | | | | 65 ± 5% RH | | | | | |
| | | | 2M | 5 ± 3° C. | 13.0 | 108.7 | 103.8 | 96.9 | 3.1 |
| | | | | 25 ± 2° C./ | 12.4 | 103.5 | 98.8 | 94.8 | 5.2 |
| | | | | 60 ± 5% RH | | | | | |
| | | | | 30 ± 2° C./ | 11.9 | 99.2 | 94.7 | 93.1 | 6.9 |
| | | | | 65 ± 5% RH | | | | | |
| | | | 3M | 5 ± 3° C. | 11.5 | 95.5 | 91.2 | 97.5 | 2.5 |
| | | | | 25 ± 2° C./ | 11.7 | 97.8 | 93.3 | 93.8 | 6.2 |
| | | | | 60 ± 5% RH | | | | | |
| | | | 6M | 5 ± 3° C. | 11.4 | 94.6 | 90.3 | 96.5 | 3.5 |
| | | | | 25 ± 2° C./ | 10.7 | 88.8 | 84.8 | 90.9 | 9.1 |
| 6 | 12 | 10 mM Tris, | Initial | N/A | 11.9 | 99.3 | 96.8 | 97.4 | 2.6 |
| | | 0.5 mg/mL | 0.5M | 30 ± 2° C./ | 11.7 | 97.9 | 95.4 | 96.1 | 3.9 |
| | | EDTA, | | 65 ± 5% RH | | | | | |
| | | 90 mg/mL | 1M | 5 ± 3° C. | 12.3 | 102.3 | 99.7 | 97.3 | 2.7 |
| | | Sucrose, | | 25 ± 2° C./ | 11.9 | 99.0 | 96.5 | 95.5 | 4.5 |
| | | pH 8.6 | | 60 ± 5% RH | | | | | |
| | | | | 30 ± 2° C./ | 11.4 | 94.6 | 92.2 | 94.3 | 5.7 |
| | | | | 65 ± 5% RH | | | | | |
| | | | 2M | 5 ± 3° C. | 13.2 | 110.3 | 107.5 | 97.0 | 3.0 |
| | | | | 25 ± 2° C./ | 11.7 | 97.6 | 95.1 | 94.1 | 5.9 |
| | | | | 60 ± 5% RH | | | | | |
| | | | | 30 ± 2° C./ | 11.9 | 99.2 | 96.6 | 92.0 | 8.0 |
| | | | | 65 ± 5% RH | | | | | |
| | | | 3M | 5 ± 3° C. | 11.4 | 94.8 | 92.4 | 97.3 | 2.7 |
| | | | | 25 ± 2° C./ | 11.4 | 95.3 | 92.9 | 93.1 | 6.9 |
| | | | | 60 ± 5% RH | | | | | |
| | | | 6M | 5 ± 3° C. | 11.4 | 95.1 | 92.7 | 96.5 | 3.5 |
| | | | | 25 ± 2° C./ | 10.4 | 86.3 | 84.1 | 89.6 | 10.4 |
| | | | | 60 ± 5% RH | | | | | |
| 7 | 12 | 10 mM Tris, | Initial | N/A | 12.2 | 101.3 | 104.9 | 97.4 | 2.6 |
| | | 0.5 mg/mL | 0.5M | 30 ± 2° C./ | 11.7 | 97.8 | 101.3 | 96.7 | 3.3 |
| | | EDTA, | | 65 ± 5% RH | | | | | |
| | | 20 mg/mL | 1M | 5 ± 3° C. | 11.9 | 99.2 | 102.8 | 97.3 | 2.7 |
| | | Propylene | | 25 ± 2° C./ | 11.6 | 96.7 | 100.2 | 96.3 | 3.7 |
| | | Glycol, | | 60 ± 5% RH | | | | | |
| | | pH 8.0 | | 30 ± 2° C./ | 11.3 | 94.6 | 97.9 | 95.8 | 4.2 |
| | | | | 65 ± 5% RH | | | | | |
| | | | 2M | 5 ± 3° C. | 12.6 | 104.6 | 108.4 | 97.1 | 2.9 |
| | | | | 25 ± 2° C./ | 11.9 | 98.8 | 102.3 | 95.6 | 4.4 |
| | | | | 60 ± 5% RH | | | | | |
| | | | | 30 ± 2° C./ | 11.8 | 98.6 | 102.1 | 94.3 | 5.7 |
| | | | | 65 ± 5% RH | | | | | |
| | | | 3M | 5 ± 3° C. | 11.3 | 93.9 | 97.2 | 97.6 | 2.4 |
| | | | | 25 ± 2° C./ | 11.6 | 96.4 | 99.8 | 95.0 | 5.0 |
| | | | | 60 ± 5% RH | | | | | |
| | | | 6M | 5 ± 3° C. | 11.3 | 93.9 | 97.2 | 96.7 | 3.3 |
| | | | | 25 ± 2° C./ | 10.8 | 90.2 | 93.5 | 93.0 | 7.0 |
| | | | | 60 ± 5% RH | | | | | |
| 8 | 12 | 10 mM Tris, | Initial | N/A | 12.0 | 100.0 | 98.3 | 97.5 | 2.5 |
| | | 0.5 mg/mL | 0.5M | 30 ± 2° C./ | 11.8 | 98.5 | 96.8 | 96.6 | 3.4 |
| | | EDTA, | | 65 ± 5% RH | | | | | |
| | | 20 mg/mL | 1M | 5 ± 3° C. | 12.1 | 100.5 | 98.8 | 97.4 | 2.6 |
| | | Propylene | | 25 ± 2° C./ | 11.9 | 99.1 | 97.5 | 96.3 | 3.7 |
| | | Glycol, | | 60 ± 5% RH | | | | | |
| | | pH 8.3 | | 30 ± 2° C./ | 11.4 | 95.4 | 93.8 | 95.1 | 4.9 |
| | | | | 65 ± 5% RH | | | | | |
| | | | 2M | 5 ± 3° C. | 12.1 | 101.1 | 99.3 | 97.0 | 3.0 |
| | | | | 25 ± 2° C./ | 12.0 | 99.6 | 97.9 | 95.1 | 4.9 |
| | | | | 60 ± 5% RH | | | | | |
| | | | | 30 ± 2° C./ | 11.9 | 95.6 | 97.8 | 93.5 | 6.5 |
| | | | | 65 ± 5% RH | | | | | |
| | | | 3M | 5 ± 3° C. | 11.5 | 95.7 | 94.1 | 97.4 | 2.6 |
| | | | | 25 ± 2° C./ | 11.4 | 95.2 | 93.6 | 94.3 | 5.7 |
| | | | | 60 ± 5% RH | | | | | |
| | | | 6M | 5 ± 3° C. | 11.5 | 95.9 | 94.3 | 97.1 | 2.9 |
| | | | | 25 ± 2° C./ | 10.6 | 88.6 | 87.1 | 91.5 | 8.5 |
| | | | | 60 ± 5% RH | | | | | |
| 9 | 12 | 10 mM Tris, | Initial | N/A | 12.1 | 101.1 | 101.2 | 97.5 | 2.5 |
| | | 0.5 mg/mL | 0.5M | 30 ± 2° C./ | 11.6 | 96.3 | 96.4 | 96.3 | 3.7 |
| | | EDTA, | | 65 ± 5% RH | | | | | |
| | | 20 mg/mL | 1M | 5 ± 3° C. | 12.0 | 100.0 | 100.1 | 97.3 | 2.7 |
| | | Propylene | | 25 ± 2° C./ | 11.6 | 97.0 | 97.1 | 95.5 | 4.5 |
| | | Glycol, | | 60 ± 5% RH | | | | | |
| | | pH 8.6 | Point | 30 ± 2° C./ | 11.3 | 94.0 | 94.1 | 94.4 | 5.6 |
| | | | | 65 ± 5% RH | | | | | |
| | | | 2M | 5 ± 3° C. | 11.8 | 98.5 | 98.6 | 96.9 | 3.1 |
| | | | | 25 ± 2° C./ | 11.9 | 98.9 | 99.0 | 94.3 | 5.7 |
| | | | | 60 ± 5% RH | | | | | |
| | | | | 30 ± 2° C./ | 11.5 | 95.6 | 95.7 | 92.3 | 7.7 |
| | | | | 65 ± 5% RH | | | | | |
| | | | 3M | 5 ± 3° C. | 12.2 | 102.0 | 102.1 | 97.1 | 2.9 |
| | | | | 25 ± 2° C./ | 11.3 | 94.4 | 94.4 | 93.2 | 6.8 |
| | | | | 60 ± 5% RH | | | | | |
| | | | 6M | 5 ± 3° C. | 11.7 | 97.5 | 97.6 | 96.4 | 3.6 |
| | | | | 25 ± 2° C./ | 10.3 | 86.2 | 86.3 | 89.4 | 10.6 |
| | | | | 60 ± 5% RH | | | | | |
| 10 | 12 | 10 mM Tris, | Initial | N/A | 11.4 | 94.7 | 100.2 | 97.4 | 2.6 |
| | | 0.5 mg/mL | 0.5M | 30 ± 2° C./ | 10.8 | 89.9 | 95.1 | 96.5 | 3.5 |
| | | EDTA, | | 65 ± 5% RH | | | | | |
| | | 46 mg/mL | 1M | 5 ± 3° C. | 11.1 | 92.7 | 98.1 | 97.3 | 2.7 |
| | | Mannitol, | | 25 ± 2° C./ | 11.0 | 91.8 | 97.1 | 96.0 | 4.0 |
| | | pH 8.3 | | 60 ± 5% RH | | | | | |
| | | with fibril | | 30 ± 2° C./ | 10.6 | 88.6 | 93.8 | 95.2 | 4.8 |
| | | seeds | | 65 ± 5% RH | | | | | |
| | | | 2M | 5 ± 3° C. | 11.1 | 92.6 | 98.0 | 96.7 | 3.3 |
| | | | | 25 ± 2° C./ | 11.1 | 92.8 | 98.2 | 95.1 | 4.9 |
| | | | | 60 ± 5% RH | | | | | |
| | | | | 30 ± 2° C./ | 11.3 | 94.0 | 99.5 | 93.4 | 6.6 |
| | | | | 65 ± 5% RH | | | | | |
| | | | 3M | 5 ± 3° C. | 10.9 | 91.2 | 96.5 | 97.4 | 2.6 |
| | | | | 25 ± 2° C./ | 11.4 | 94.7 | 100.2 | 94.5 | 5.5 |
| | | | | 60 ± 5% RH | | | | | |
| | | | 6M | 5 ± 3° C. | 10.8 | 90.3 | 95.5 | 97.2 | 2.8 |
| | | | | 25 ± 2° C./ | 10.3 | 85.7 | 90.6 | 92.1 | 7.9 |
| | | | | 60 ± 5% RH | | | | | |
| 11 | 12 | 10 mM Tris, | Initial | N/A | 11.8 | 98.0 | 96.8 | 97.5 | 2.5 |
| | | 0.5 mg/mL | 0.5M | 30 ± 2° C./ | 12.1 | 100.5 | 99.2 | 96.3 | 3.7 |
| | | EDTA, | | 65 ± 5% RH | | | | | |
| | | 90 mg/mL | 1M | 5 ± 3° C. | 12.1 | 100.7 | 99.5 | 97.3 | 2.7 |
| | | Sucrose, | | 25 ± 2° C./ | 12.1 | 101.2 | 100.0 | 95.7 | 4.3 |
| | | pH 8.3 | | 60 ± 5% RH | | | | | |
| | | with fibril | | 30 ± 2° C./ | 11.5 | 95.5 | 94.3 | 94.7 | 5.3 |
| | | seeds | | 65 ± 5% RH | | | | | |
| | | | 2M | 5 ± 3° C. | 12.4 | 103.3 | 102.1 | 96.9 | 3.1 |
| | | | | 25 ± 2° C./ | 11.6 | 96.8 | 95.6 | 94.6 | 5.4 |
| | | | | 60 ± 5% RH | | | | | |
| | | | | 30 ± 2° C./ | 12.0 | 100.4 | 99.1 | 92.8 | 7.2 |
| | | | | 65 ± 5% RH | | | | | |
| | | | 3M | 5 ± 3° C. | 11.7 | 97.6 | 96.4 | 97.3 | 2.7 |
| | | | | 25 ± 2° C./ | 11.6 | 96.7 | 95.5 | 94.0 | 6.0 |
| | | | | 60 ± 5% RH | | | | | |
| | | | 6M | 5 ± 3° C. | 11.5 | 96.1 | 94.9 | 96.9 | 3.1 |
| | | | | 25 ± 2° C./ | 10.6 | 88.7 | 87.6 | 90.9 | 9.1 |
| | | | | 60 ± 5% RH | | | | | |
| 12 | 12 | 10 mM Tris, | Initial | N/A | 11.8 | 98.3 | 98.6 | 97.4 | 2.6 |
| | | 0.5 mg/mL | 0.5M | 30 ± 2° C./ | 11.7 | 97.8 | 98.1 | 96.5 | 3.5 |
| | | EDTA, | | 65 ± 5% RH | | | | | |
| | | 20 mg/mL | 1M | 5 ± 3° C. | 12.0 | 100.0 | 100.4 | 97.2 | 2.8 |
| | | Propylene | | 25 ± 2° C./ | 11.8 | 98.2 | 98.6 | 96.0 | 4.0 |
| | | Glycol, | | 60 ± 5% RH | | | | | |
| | | pH 8.3 | | 30 ± 2° C./ | 11.5 | 95.5 | 95.9 | 94.9 | 5.1 |
| | | with fibril | | 65 ± 5% RH | | | | | |
| | | seeds | 2M | 5 ± 3° C. | 11.8 | 98.1 | 98.4 | 97.0 | 3.0 |
| | | | | 25 ± 2° C./ | 11.9 | 99.3 | 99.6 | 94.8 | 5.2 |
| | | | | 60 ± 5% RH | | | | | |
| | | | | 30 ± 2° C./ | 11.6 | 96.9 | 97.3 | 93.4 | 6.6 |
| | | | | 65 ± 5% RH | | | | | |
| | | | 3M | 5 ± 3° C. | 11.7 | 97.1 | 97.4 | 97.2 | 2.8 |
| | | | | 25 ± 2° C./ | 11.4 | 94.9 | 95.2 | 94.3 | 5.7 |
| | | | | 60 ± 5% RH | | | | | |
| | | | 6M | 5 ± 3° C. | 11.4 | 94.8 | 95.1 | 96.9 | 3.1 |
| | | | | 25 ± 2° C./ | 10.8 | 89.9 | 90.2 | 92.0 | 8.0 |
| | | | | 60 ± 5% RH | | | | | |

### (c) Size Exclusion Chromatography

Results for the analysis of purity by SEC are shown in Table 12. Across the set of formulations, the initial monomer percentage ranged from 98.8-99.2%, the percentage of aggregates for all formulations was 0.3%, and the percentage of fragments ranged from 0.5-0.9%. Across the set of formulations at the 6M/5±3° C. time point and conditions, the monomer percentage ranged from 98.7-99.0%, the percentage of aggregates ranged from 0.4-0.5%, and the percentage of fragments ranged from 0.6-0.9%. Across the set of formulations at the 6M/25±2° C./60±5% RH time point and conditions, the monomer percentage ranged from 98.2-98.7%, the percentage of aggregates ranged from 0.7-1.0%, and the percentage of fragments ranged from 0.6-1.0%. Across the set of formulations at the 2M/30±2° C./65±5% RH time point and conditions, the monomer percentage ranged from 98.4-99.0%, the percentage of aggregates ranged from 0.5-0.8%, and the percentage of fragments ranged from 0.5-1.0%. For each formulation, similar patterns of decline in percentage of monomer and increases in the percentage of aggregates and fragments were observed across the stability conditions involved. While the marginal changes may be due to method variability, the sucrose-based formulations demonstrated the most change in percent monomer whereas mannitol-based formulations demonstrated the least change in percent monomer. In addition, higher pH values demonstrated the most change for each excipient type, with exception to propylene glycol. Furthermore, when comparing the same formulation components with and without fibril seeds, overall those with fibril seeds demonstrated a higher purity, especially at the accelerated conditions.

Overall, the data shows minimal aggregate and fragment changes after 6 months of storage at accelerated conditions.

TABLE 12
Summary of SEC Results

| | | | | | | Total | |
|---|---|---|---|---|---|---|---|
| | | | | | Mon- | Ag- | Total |
| | API | | Time | | omer | gregates | Fragments |
| ID | (mg/mL) | Composition | Point | Condition | % | % | % |
| 1 | 12 | 10 mM Tris, | Initial | N/A | 98.9 | 0.3 | 0.8 |
| | | 0.5 mg/mL | 0.5M | 30 ± 2° C./65 ± 5% RH | 98.8 | 0.4 | 0.7 |
| | | EDTA, | 1M | 5 ± 3° C. | 98.7 | 0.4 | 0.9 |
| | | 46 mg/mL | | 25 ± 2° C./60 ± 5% RH | 98.6 | 0.5 | 1.0 |
| | | Mannitol, | | 30 ± 2° C./65 ± 5% RH | 98.5 | 0.5 | 1.0 |
| | | pH 8.0 | 2M | 5 ± 3° C. | 98.7 | 0.4 | 1.0 |
| | | | | 25 ± 2° C./60 ± 5% RH | 98.7 | 0.5 | 0.8 |
| | | | | 30 ± 2° C./65 ± 5% RH | 98.4 | 0.6 | 1.0 |
| | | | 3M | 5 ± 3° C. | 98.8 | 0.4 | 0.8 |
| | | | | 25 ± 2° C./60 ± 5% RH | 98.6 | 0.5 | 0.8 |
| | | | 6M | 5 ± 3° C. | 98.7 | 0.4 | 0.9 |
| | | | | 25 ± 2° C./60 ± 5% RH | 98.4 | 0.7 | 0.9 |
| 2 | 12 | 10 mM Tris, | Initial | N/A | 98.8 | 0.3 | 0.9 |
| | | 0.5 mg/mL | 0.5M | 30 ± 2° C./65 ± 5% RH | 98.7 | 0.4 | 0.9 |
| | | EDTA, | 1M | 5 ± 3° C. | 98.7 | 0.4 | 1.0 |
| | | 46 mg/mL | | 25 ± 2° C./60 ± 5% RH | 98.5 | 0.4 | 1.0 |
| | | Mannitol, | | 30 ± 2° C./65 ± 5% RH | 98.4 | 0.5 | 1.1 |
| | | pH 8.3 | 2M | 5 ± 3° C. | 98.7 | 0.4 | 0.9 |
| | | | | 25 ± 2° C./60 ± 5% RH | 98.6 | 0.5 | 0.9 |
| | | | | 30 ± 2° C./65 ± 5% RH | 98.6 | 0.5 | 0.8 |
| | | | 3M | 5 ± 3° C. | 98.9 | 0.4 | 0.8 |
| | | | | 25 ± 2° C./60 ± 5% RH | 98.8 | 0.5 | 0.8 |
| | | | 6M | 5 ± 3° C. | 98.7 | 0.4 | 0.9 |
| | | | | 25 ± 2° C./60 ± 5% RH | 98.4 | 0.7 | 1.0 |
| 3 | 12 | 10 mM Tris, | Initial | N/A | 98.9 | 0.3 | 0.8 |
| | | 0.5 mg/mL | 0.5M | 30 ± 2° C./65 ± 5% RH | 98.8 | 0.4 | 0.8 |
| | | EDTA, | 1M | 5 ± 3° C. | 98.6 | 0.4 | 1.0 |
| | | 46 mg/mL | | 25 ± 2° C./60 ± 5% RH | 98.5 | 0.4 | 1.1 |
| | | Mannitol, | | 30 ± 2° C./65 ± 5% RH | 98.5 | 0.5 | 1.0 |
| | | pH 8.6 | 2M | 5 ± 3° C. | 98.9 | 0.4 | 0.8 |
| | | | | 25 ± 2° C./60 ± 5% RH | 98.7 | 0.5 | 0.8 |
| | | | | 30 ± 2° C./65 ± 5% RH | 98.7 | 0.5 | 0.8 |
| | | | 3M | 5 ± 3° C. | 99.0 | 0.3 | 0.7 |
| | | | | 25 ± 2° C./60 ± 5% RH | 98.8 | 0.5 | 0.7 |
| | | | 6M | 5 ± 3° C. | 98.7 | 0.4 | 0.9 |
| | | | | 25 ± 2° C./60 ± 5% RH | 98.4 | 0.7 | 1.0 |
| 4 | 12 | 10 mM Tris, | Initial | N/A | 99.0 | 0.3 | 0.7 |
| | | 0.5 mg/mL | 0.5M | 30 ± 2° C./65 ± 5% RH | 98.9 | 0.5 | 0.6 |
| | | EDTA, | 1M | 5 ± 3° C. | 98.6 | 0.4 | 1.0 |
| | | 90 mg/mL | | 25 ± 2° C./60 ± 5% RH | 98.4 | 0.5 | 1.0 |
| | | Sucrose, | | 30 ± 2° C./65 ± 5% RH | 98.4 | 0.6 | 1.0 |
| | | pH 8.0 | 2M | 5 ± 3° C. | 98.9 | 0.4 | 0.7 |
| | | | | 25 ± 2° C./60 ± 5% RH | 98.7 | 0.6 | 0.7 |
| | | | | 30 ± 2° C./65 ± 5% RH | 98.5 | 0.8 | 0.7 |
| | | | 3M | 5 ± 3° C. | 99.0 | 0.4 | 0.6 |
| | | | | 25 ± 2° C./60 ± 5% RH | 98.7 | 0.7 | 0.6 |
| | | | 6M | 5 ± 3° C. | 98.8 | 0.5 | 0.8 |
| | | | | 25 ± 2° C./60 ± 5% RH | 98.2 | 1.0 | 0.8 |
| 5 | 12 | 10 mM Tris, | Initial | N/A | 99.0 | 0.3 | 0.6 |
| | | 0.5 mg/mL | 0.5M | 30 ± 2° C./65 ± 5% RH | 98.9 | 0.5 | 0.6 |
| | | EDTA, | 1M | 5 ± 3° C. | 98.6 | 0.4 | 1.1 |
| | | 90 mg/mL | | 25 ± 2° C./60 ± 5% RH | 98.4 | 0.5 | 1.1 |
| | | Sucrose, | | 30 ± 2° C./65 ± 5% RH | 98.3 | 0.6 | 1.1 |
| | | pH 8.3 | 2M | 5 ± 3° C. | 98.9 | 0.4 | 0.7 |
| | | | | 25 ± 2° C./60 ± 5% RH | 98.8 | 0.6 | 0.6 |
| | | | | 30 ± 2° C./65 ± 5% RH | 98.7 | 0.7 | 0.6 |
| | | | 3M | 5 ± 3° C. | 99.0 | 0.4 | 0.6 |
| | | | | 25 ± 2° C./60 ± 5% RH | 98.7 | 0.6 | 0.6 |
| | | | 6M | 5 ± 3° C. | 98.8 | 0.5 | 0.7 |
| | | | | 25 ± 2° C./60 ± 5% RH | 98.3 | 0.9 | 0.8 |
| 6 | 12 | 10 mM Tris, | Initial | N/A | 99.1 | 0.3 | 0.6 |
| | | 0.5 mg/mL | 0.5M | 30 ± 2° C./65 ± 5% RH | 98.9 | 0.4 | 0.6 |
| | | EDTA, | 1M | 5 ± 3° C. | 98.6 | 0.4 | 1.0 |
| | | 90 mg/mL | | 25 ± 2° C./60 ± 5% RH | 98.4 | 0.5 | 1.1 |
| | | Sucrose, | | 30 ± 2° C./65 ± 5% RH | 98.4 | 0.5 | 1.0 |
| | | pH 8.6 | 2M | 5 ± 3° C. | 99.0 | 0.4 | 0.6 |
| | | | | 25 ± 2° C./60 ± 5% RH | 98.9 | 0.6 | 0.5 |
| | | | | 30 ± 2° C./65 ± 5% RH | 98.7 | 0.7 | 0.6 |
| | | | 3M | 5 ± 3° C. | 99.0 | 0.4 | 0.6 |
| | | | | 25 ± 2° C./60 ± 5% RH | 98.8 | 0.6 | 0.6 |
| | | | 6M | 5 ± 3° C. | 98.9 | 0.4 | 0.7 |
| | | | | 25 ± 2° C./60 ± 5% RH | 98.3 | 1.0 | 0.7 |
| 7 | 12 | 10 mM Tris, | Initial | N/A | 99.1 | 0.3 | 0.6 |
| | | 0.5 mg/mL EDTA, | 0.5M | 30 ± 2° C./65 ± 5% RH | 99.1 | 0.4 | 0.6 |
| | | 20 mg/mL | 1M | 5 ± 3° C. | 98.6 | 0.3 | 1.0 |
| | | Propylene Glycol, | | 25 ± 2° C./60 ± 5% RH | 98.5 | 0.4 | 1.1 |
| | | pH 8.0 | | 30 ± 2° C./65 ± 5% RH | 98.4 | 0.5 | 1.1 |
| | | | 2M | 5 ± 3° C. | 99.1 | 0.4 | 0.5 |
| | | | | 25 ± 2° C./60 ± 5% RH | 98.9 | 0.5 | 0.6 |
| | | | | 30 ± 2° C./65 ± 5% RH | 98.8 | 0.6 | 0.6 |
| | | | 3M | 5 ± 3° C. | 99.1 | 0.3 | 0.6 |
| | | | | 25 ± 2° C./60 ± 5% RH | 98.9 | 0.5 | 0.6 |
| | | | 6M | 5 ± 3° C. | 98.9 | 0.4 | 0.6 |
| | | | | 25 ± 2° C./60 ± 5% RH | 98.6 | 0.7 | 0.7 |
| 8 | 12 | 10 mM Tris, | Initial | N/A | 99.1 | 0.3 | 0.6 |
| | | 0.5 mg/mL EDTA, | 0.5M | 30 ± 2° C./65 ± 5% RH | 99.0 | 0.4 | 0.7 |
| | | 20 mg/mL | 1M | 5 ± 3° C. | 98.5 | 0.3 | 1.1 |
| | | Propylene Glycol, | | 25 ± 2° C./60 ± 5% RH | 98.5 | 0.4 | 1.1 |
| | | pH 8.3 | | 30 ± 2° C./65 ± 5% RH | 98.4 | 0.5 | 1.1 |
| | | | 2M | 5 ± 3° C. | 99.1 | 0.4 | 0.5 |
| | | | | 25 ± 2° C./60 ± 5% RH | 99.0 | 0.5 | 0.5 |
| | | | | 30 ± 2° C./65 ± 5% RH | 98.9 | 0.6 | 0.5 |
| | | | 3M | 5 ± 3° C. | 99.1 | 0.4 | 0.6 |
| | | | | 25 ± 2° C./60 ± 5% RH | 98.9 | 0.5 | 0.6 |
| | | | 6M | 5 ± 3° C. | 99.0 | 0.4 | 0.6 |
| | | | | 25 ± 2° C./60 ± 5% RH | 98.6 | 0.7 | 0.7 |
| 9 | 12 | 10 mM Tris, | Initial | N/A | 99.1 | 0.3 | 0.6 |
| | | 0.5 mg/mL | 0.5M | 30 ± 2° C./65 ± 5% RH | 99.0 | 0.4 | 0.6 |
| | | EDTA, | 1M | 5 ± 3° C. | 98.5 | 0.4 | 1.1 |
| | | 20 mg/mL | | 25 ± 2° C./60 ± 5% RH | 98.5 | 0.4 | 1.1 |
| | | Propylene | | 30 ± 2° C./65 ± 5% RH | 98.4 | 0.4 | 1.1 |
| | | Glycol, pH 8.6 | 2M | 5 ± 3° C. | 99.2 | 0.4 | 0.5 |
| | | | | 25 ± 2° C./60 ± 5% RH | 99.0 | 0.5 | 0.5 |
| | | | | 30 ± 2° C./65 ± 5% RH | 99.0 | 0.6 | 0.5 |
| | | | 3M | 5 ± 3° C. | 99.1 | 0.3 | 0.6 |
| | | | | 25 ± 2° C./60 ± 5% RH | 98.9 | 0.5 | 0.6 |
| | | | 6M | 5 ± 3° C. | 99.0 | 0.4 | 0.6 |
| | | | | 25 ± 2° C./60 ± 5% RH | 98.6 | 0.7 | 0.7 |
| 10 | 12 | 10 mM Tris, | Initial | N/A | 99.1 | 0.3 | 0.6 |
| | | 0.5 mg/mL | 0.5M | 30 ± 2° C./65 ± 5% RH | 98.9 | 0.4 | 0.7 |
| | | EDTA, | 1M | 5 ± 3° C. | 98.7 | 0.2 | 1.1 |
| | | 46 mg/mL | | 25 ± 2° C./60 ± 5% RH | 98.4 | 0.4 | 1.2 |
| | | Mannitol, | | 30 ± 2° C./65 ± 5% RH | 98.3 | 0.5 | 1.2 |
| | | pH 8.3 with | 2M | 5 ± 3° C. | 99.1 | 0.4 | 0.5 |
| | | fibril seeds | | 25 ± 2° C./60 ± 5% RH | 99.0 | 0.5 | 0.5 |
| | | | | 30 ± 2° C./65 ± 5% RH | 98.9 | 0.6 | 0.5 |
| | | | 3M | 5 ± 3° C. | 99.1 | 0.4 | 0.6 |
| | | | Point | 25 ± 2° C./60 ± 5% RH | 98.9 | 0.5 | 0.6 |
| | | | 6M | 5 ± 3° C. | 99.0 | 0.4 | 0.6 |
| | | | | 25 ± 2° C./60 ± 5% RH | 98.6 | 0.7 | 0.7 |
| 11 | 12 | 10 mM Tris, | Initial | N/A | 99.1 | 0.3 | 0.5 |
| | | 0.5 mg/mL | 0.5M | 30 ± 2° C./65 ± 5% RH | 98.9 | 0.5 | 0.6 |
| | | EDTA, | 1M | 5 ± 3° C. | 98.7 | 0.4 | 1.0 |
| | | 90 mg/mL | | 25 ± 2° C./60 ± 5% RH | 98.6 | 0.5 | 0.9 |
| | | Sucrose, | | 30 ± 2° C./65 ± 5% RH | 98.6 | 0.6 | 0.9 |
| | | pH 8.3 with | 2M | 5 ± 3° C. | 99.1 | 0.4 | 0.5 |
| | | fibril seeds | | 25 ± 2° C./60 ± 5% RH | 98.9 | 0.6 | 0.5 |
| | | | | 30 ± 2° C./65 ± 5% RH | 98.8 | 0.7 | 0.5 |
| | | | 3M | 5 ± 3° C. | 99.1 | 0.4 | 0.5 |
| | | | | 25 ± 2° C./60 ± 5% RH | 98.8 | 0.6 | 0.6 |
| | | | 6M | 5 ± 3° C. | 99.0 | 0.5 | 0.6 |
| | | | | 25 ± 2° C./60 ± 5% RH | 98.4 | 0.9 | 0.7 |
| 12 | 12 | 10 mM Tris, | Initial | N/A | 99.2 | 0.3 | 0.5 |
| | | 0.5 mg/mL | 0.5M | 30 ± 2° C./65 ± 5% RH | 99.1 | 0.4 | 0.5 |
| | | EDTA, | 1M | 5 ± 3° C. | 98.8 | 0.3 | 0.8 |
| | | 20 mg/mL | | 25 ± 2° C./60 ± 5% RH | 98.8 | 0.4 | 0.8 |
| | | Propylene | | 30 ± 2° C./65 ± 5% RH | 98.7 | 0.5 | 0.8 |
| | | Glycol, | 2M | 5 ± 3° C. | 99.1 | 0.4 | 0.5 |
| | | pH 8.3 with | | 25 ± 2° C./60 ± 5% RH | 99.0 | 0.5 | 0.5 |
| | | fibril seeds | | 30 ± 2° C./65 ± 5% RH | 98.9 | 0.5 | 0.5 |
| | | | 3M | 5 ± 3° C. | 99.1 | 0.4 | 0.5 |
| | | | | 25 ± 2° C./60 ± 5% RH | 98.9 | 0.5 | 0.6 |
| | | | 6M | 5 ± 3° C. | 99.0 | 0.4 | 0.6 |
| | | | | 25 ± 2° C./60 ± 5% RH | 98.7 | 0.7 | 0.6 |

### (d) Anion Exchange Chromatography

The results for charge heterogeneity by AEX are shown in Table 13. Across the set of formulations, the initial main peak percentage ranged from 98.1-98.7%, the percentage of basic variants ranged from 0.6-1.1%, and the percentage of acidic variants ranged from 0.6-0.8%. Across the set of formulations at the 6M/5±3° C. time point and conditions, the main peak percentage ranged from 93.3-97.1%, the percentage of basic variants ranged from 0.5-0.9%, and the percentage of acidic variants ranged from 2.3-6.0%. Across the set of formulations at the 6M/25±2° C./60±5% RH time point and conditions, the main peak percentage ranged from 86.5-91.6%, the percentage of basic variants ranged from 0.9-1.9%, and the percentage of acidic variants ranged from 7.3-11.8%. Across the set of formulations at the 2M/30±2° C./65±5% RH time point and conditions, the main peak percentage ranged from 92.4-93.3%, the percentage of basic variants ranged from 1.0-1.4%, and the percentage of acidic variants ranged from 5.3-6.5%. For each formulation, similar patterns of decline in percentage of main peak and increases in the percentage of acidic and basic variants were observed across the stability conditions involved.

TABLE 13
Summary of AEX Results

| | | | | | | Total | Total |
|---|---|---|---|---|---|---|---|
| | | | | | Main | Basic | Acidic |
| | API | | Time | | Peak | Variants | Variants |
| ID | (mg/mL) | Composition | Point | Condition | % | % | % |
| 1 | 12 | 10 mM Tris, | Initial | N/A | 98.6 | 0.8 | 0.6 |
| | | 0.5 mg/mL | 0.5M | 30 ± 2° C./65 ± 5% RH | 97.1 | 1.2 | 1.7 |
| | | EDTA, | 1M | 5 ± 3° C. | 97.8 | 1.4 | 0.8 |
| | | 46 mg/mL | | 25 ± 2° C./60 ± 5% RH | 97.0 | 1.1 | 1.9 |
| | | Mannitol, | | 30 ± 2° C./65 ± 5% RH | 96.4 | 1.0 | 2.5 |
| | | pH 8.0 | 2M | 5 ± 3° C. | 97.5 | 1.3 | 1.2 |
| | | | | 25 ± 2° C./60 ± 5% RH | 95.5 | 1.1 | 3.5 |
| | | | | 30 ± 2° C./65 ± 5% RH | 93.2 | 1.1 | 5.6 |
| | | | 3M | 5 ± 3° C. | 96.8 | 1.3 | 1.9 |
| | | | | 25 ± 2° C./60 ± 5% RH | 93.3 | 1.3 | 5.4 |
| | | | 6M | 5 ± 3° C. | 95.2 | 0.9 | 3.9 |
| | | | | 25 ± 2° C./60 ± 5% RH | 90.1 | 1.3 | 8.5 |
| 2 | 12 | 10 mM Tris, | Initial | N/A | 98.6 | 0.8 | 0.6 |
| | | 0.5 mg/mL | 0.5M | 30 ± 2° C./65 ± 5% RH | 97.1 | 1.1 | 1.8 |
| | | EDTA, | 1M | 5 ± 3° C. | 98.1 | 1.0 | 0.9 |
| | | 46 mg/mL | | 25 ± 2° C./60 ± 5% RH | 96.9 | 0.9 | 2.2 |
| | | Mannitol, | | 30 ± 2° C./65 ± 5% RH | 96.3 | 0.9 | 2.8 |
| | | pH 8.3 | 2M | 5 ± 3° C. | 98.5 | 0.9 | 0.6 |
| | | | | 25 ± 2° C./60 ± 5% RH | 94.6 | 1.2 | 4.2 |
| | | | | 30 ± 2° C./65 ± 5% RH | 93.0 | 1.2 | 5.8 |
| | | | 3M | 5 ± 3° C. | 96.7 | 1.2 | 2.0 |
| | | | | 25 ± 2° C./60 ± 5% RH | 93.2 | 1.2 | 5.6 |
| | | | 6M | 5 ± 3° C. | 95.9 | 0.9 | 3.2 |
| | | | | 25 ± 2° C./60 ± 5% RH | 89.8 | 1.2 | 9.0 |
| 3 | 12 | 10 mM Tris, | Initial | N/A | 98.7 | 0.7 | 0.6 |
| | | 0.5 mg/mL | 0.5M | 30 ± 2° C./65 ± 5% RH | 97.0 | 1.0 | 2.0 |
| | | EDTA, | 1M | 5 ± 3° C. | 98.1 | 0.9 | 0.9 |
| | | 46 mg/mL | | 25 ± 2° C./60 ± 5% RH | 97.2 | 0.8 | 2.0 |
| | | Mannitol, | | 30 ± 2° C./65 ± 5% RH | 96.1 | 0.9 | 3.0 |
| | | pH 8.6 | 2M | 5 ± 3° C. | 97.9 | 0.8 | 1.3 |
| | | | | 25 ± 2° C./60 ± 5% RH | 93.9 | 2.2 | 3.9 |
| | | | Point | 30 ± 2° C./65 ± 5% RH | 92.5 | 1.4 | 6.1 |
| | | | 3M | 5 ± 3° C. | 96.2 | 1.2 | 2.6 |
| | | | | 25 ± 2° C./60 ± 5% RH | 93.2 | 1.4 | 5.5 |
| | | | 6M | 5 ± 3° C. | 95.2 | 0.7 | 4.1 |
| | | | | 25 ± 2° C./60 ± 5% RH | 88.9 | 1.0 | 10.1 |
| 4 | 12 | 10 mM Tris, | Initial | N/A | 98.6 | 0.7 | 0.7 |
| | | 0.5 mg/mL | 0.5M | 30 ± 2° C./65 ± 5% RH | 97.1 | 1.1 | 1.8 |
| | | EDTA, | 1M | 5 ± 3° C. | 98.0 | 1.0 | 1.0 |
| | | 90 mg/mL | | 25 ± 2° C./60 ± 5% RH | 97.2 | 0.9 | 1.9 |
| | | Sucrose, | | 30 ± 2° C./65 ± 5% RH | 96.2 | 1.1 | 2.7 |
| | | pH 8.0 | 2M | 5 ± 3° C. | 98.2 | 1.2 | 0.7 |
| | | | | 25 ± 2° C./60 ± 5% RH | 95.6 | 1.2 | 3.1 |
| | | | | 30 ± 2° C./65 ± 5% RH | 93.1 | 1.3 | 5.6 |
| | | | 3M | 5 ± 3° C. | 96.4 | 1.2 | 2.4 |
| | | | | 25 ± 2° C./60 ± 5% RH | 94.2 | 1.3 | 4.5 |
| | | | 6M | 5 ± 3° C. | 93.3 | 0.7 | 6.0 |
| | | | | 25 ± 2° C./60 ± 5% RH | 88.5 | 1.2 | 10.4 |
| 5 | 12 | 10 mM Tris, | Initial | N/A | 98.7 | 0.6 | 0.7 |
| | | 0.5 mg/mL | 0.5M | 30 ± 2° C./65 ± 5% RH | 97.0 | 1.0 | 1.9 |
| | | EDTA, | 1M | 5 ± 3° C. | 98.2 | 1.0 | 0.8 |
| | | 90 mg/mL | | 25 ± 2° C./60 ± 5% RH | 97.0 | 0.9 | 2.1 |
| | | Sucrose, | | 30 ± 2° C./65 ± 5% RH | 96.1 | 0.9 | 3.0 |
| | | pH 8.3 | 2M | 5 ± 3° C. | 97.7 | 1.2 | 1.1 |
| | | | | 25 ± 2° C./60 ± 5% RH | 95.1 | 1.0 | 3.8 |
| | | | | 30 ± 2° C./65 ± 5% RH | 92.6 | 1.1 | 6.3 |
| | | | 3M | 5 ± 3° C. | 96.5 | 1.1 | 2.4 |
| | | | | 25 ± 2° C./60 ± 5% RH | 92.4 | 1.2 | 6.4 |
| | | | 6M | 5 ± 3° C. | 95.6 | 0.9 | 3.5 |
| | | | | 25 ± 2° C./60 ± 5% RH | 89.4 | 1.0 | 9.6 |
| 6 | 12 | 10 mM Tris, | Initial | N/A | 98.6 | 0.6 | 0.7 |
| | | 0.5 mg/mL | 0.5M | 30 ± 2° C./65 ± 5% RH | 96.9 | 1.1 | 2.0 |
| | | EDTA, | 1M | 5 ± 3° C. | 98.2 | 1.0 | 0.8 |
| | | 90 mg/mL | | 25 ± 2° C./60 ± 5% RH | 96.7 | 0.9 | 2.4 |
| | | Sucrose, | | 30 ± 2° C./65 ± 5% RH | 95.9 | 0.9 | 3.3 |
| | | pH 8.6 | 2M | 5 ± 3° C. | 97.3 | 1.4 | 1.3 |
| | | | | 25 ± 2° C./60 ± 5% RH | 95.6 | 1.0 | 3.4 |
| | | | | 30 ± 2° C./65 ± 5% RH | 92.9 | 1.0 | 6.1 |
| | | | 3M | 5 ± 3° C. | 97.2 | 0.8 | 2.0 |
| | | | | 25 ± 2° C./60 ± 5% RH | 92.4 | 1.1 | 6.5 |
| | | | 6M | 5 ± 3° C. | 95.7 | 0.5 | 3.8 |
| | | | | 25 ± 2° C./60 ± 5% RH | 89.3 | 0.9 | 9.8 |
| 7 | 12 | 10 mM Tris, | Initial | N/A | 98.1 | 1.1 | 0.8 |
| | | 0.5 mg/mL | 0.5M | 30 ± 2° C./65 ± 5% RH | 97.2 | 1.0 | 1.8 |
| | | EDTA, | 1M | 5 ± 3° C. | 98.2 | 0.9 | 0.9 |
| | | 20 mg/mL | | 25 ± 2° C./60 ± 5% RH | 96.9 | 0.9 | 2.1 |
| | | Propylene | | 30 ± 2° C./65 ± 5% RH | 96.4 | 1.0 | 2.7 |
| | | Glycol, pH 8.0 | 2M | 5 ± 3° C. | 98.0 | 0.9 | 1.1 |
| | | | | 25 ± 2° C./60 ± 5% RH | 95.1 | 0.9 | 3.9 |
| | | | | 30 ± 2° C./65 ± 5% RH | 93.3 | 1.1 | 5.6 |
| | | | 3M | 5 ± 3° C. | 96.9 | 1.1 | 2.0 |
| | | | | 25 ± 2° C./60 ± 5% RH | 93.8 | 1.1 | 5.1 |
| | | | 6M | 5 ± 3° C. | 97.1 | 0.7 | 2.3 |
| | | | | 25 ± 2° C./60 ± 5% RH | 91.6 | 1.2 | 7.3 |
| 8 | 12 | 10 mM Tris, | Initial | N/A | 98.3 | 1.0 | 0.7 |
| | | 0.5 mg/mL | 0.5M | 30 ± 2° C./65 ± 5% RH | 97.4 | 0.7 | 1.9 |
| | | EDTA, | 1M | 5 ± 3° C. | 98.3 | 0.9 | 0.8 |
| | | 20 mg/mL | | 25 ± 2° C./60 ± 5% RH | 97.0 | 0.8 | 2.2 |
| | | Propylene | | 30 ± 2° C./65 ± 5% RH | 96.3 | 0.9 | 2.7 |
| | | Glycol, pH 8.3 | 2M | 5 ± 3° C. | 98.2 | 1.0 | 0.9 |
| | | | | 25 ± 2° C./60 ± 5% RH | 94.7 | 1.0 | 4.3 |
| | | | | 30 ± 2° C./65 ± 5% RH | 93.1 | 1.2 | 5.7 |
| | | | 3M | 5 ± 3° C. | 97.5 | 0.6 | 1.9 |
| | | | | 25 ± 2° C./60 ± 5% RH | 92.9 | 1.6 | 5.5 |
| | | | 6M | 5 ± 3° C. | 95.5 | 0.6 | 3.9 |
| | | | Point | 25 ± 2° C./60 ± 5% RH | 87.5 | 1.9 | 10.6 |
| 9 | 12 | 10 mM Tris, | Initial | N/A | 98.5 | 0.8 | 0.7 |
| | | 0.5 mg/mL | 0.5M | 30 ± 2° C./65 ± 5% RH | 96.9 | 1.0 | 2.1 |
| | | EDTA, | 1M | 5 ± 3° C. | 98.5 | 0.8 | 0.7 |
| | | 20 mg/mL | | 25 ± 2° C./60 ± 5% RH | 96.9 | 0.8 | 2.3 |
| | | Propylene | | 30 ± 2° C./65 ± 5% RH | 96.1 | 0.8 | 3.0 |
| | | Glycol, | 2M | 5 ± 3° C. | 97.9 | 0.9 | 1.2 |
| | | pH 8.6 | | 25 ± 2° C./60 ± 5% RH | 94.5 | 0.9 | 4.6 |
| | | | | 30 ± 2° C./65 ± 5% RH | 93.0 | 1.2 | 5.9 |
| | | | 3M | 5 ± 3° C. | 97.1 | 1.1 | 1.8 |
| | | | | 25 ± 2° C./60 ± 5% RH | 92.6 | 1.2 | 6.2 |
| | | | 6M | 5 ± 3° C. | 96.1 | 0.7 | 3.3 |
| | | | | 25 ± 2° C./60 ± 5% RH | 90.3 | 0.9 | 8.8 |
| 10 | 12 | 10 mM Tris, | Initial | N/A | 98.5 | 0.9 | 0.6 |
| | | 0.5 mg/mL | 0.5M | 30 ± 2° C./65 ± 5% RH | 97.0 | 1.0 | 2.0 |
| | | EDTA, | 1M | 5 ± 3° C. | 98.3 | 0.9 | 0.8 |
| | | 46 mg/mL | | 25 ± 2° C./60 ± 5% RH | 97.2 | 0.8 | 2.1 |
| | | Mannitol, | | 30 ± 2° C./65 ± 5% RH | 96.2 | 0.8 | 3.1 |
| | | pH 8.3 with | 2M | 5 ± 3° C. | 97.5 | 1.1 | 1.4 |
| | | fibril seeds | | 25 ± 2° C./60 ± 5% RH | 95.0 | 1.0 | 4.0 |
| | | | | 30 ± 2° C./65 ± 5% RH | 92.7 | 1.1 | 6.2 |
| | | | 3M | 5 ± 3° C. | 97.0 | 1.2 | 1.9 |
| | | | | 25 ± 2° C./60 ± 5% RH | 92.7 | 1.2 | 6.1 |
| | | | 6M | 5 ± 3° C. | 96.8 | 0.7 | 2.5 |
| | | | | 25 ± 2° C./60 ± 5% RH | 90.8 | 1.0 | 8.2 |
| 11 | 12 | 10 mM Tris, | Initial | N/A | 98.6 | 0.8 | 0.6 |
| | | 0.5 mg/mL | 0.5M | 30 ± 2° C./65 ± 5% RH | 97.1 | 0.8 | 2.1 |
| | | EDTA, | 1M | 5 ± 3° C. | 98.2 | 0.8 | 1.0 |
| | | 90 mg/mL | | 25 ± 2° C./60 ± 5% RH | 96.8 | 0.9 | 2.3 |
| | | Sucrose, | | 30 ± 2° C./65 ± 5% RH | 95.9 | 1.0 | 3.1 |
| | | pH 8.3 with | 2M | 5 ± 3° C. | 97.9 | 1.1 | 1.1 |
| | | fibril seeds | | 25 ± 2° C./60 ± 5% RH | 94.6 | 1.0 | 4.4 |
| | | | Point | 30 ± 2° C./65 ± 5% RH | 92.4 | 1.2 | 6.5 |
| | | | 3M | 5 ± 3° C. | 96.1 | 1.1 | 2.7 |
| | | | | 25 ± 2° C./60 ± 5% RH | 93.0 | 1.1 | 6.0 |
| | | | 6M | 5 ± 3° C. | 95.6 | 0.6 | 3.9 |
| | | | | 25 ± 2° C./60 ± 5% RH | 86.5 | 1.8 | 11.8 |
| 12 | 12 | 10 mM Tris, | Initial | N/A | 98.6 | 0.7 | 0.7 |
| | | 0.5 mg/mL | 0.5M | 30 ± 2° C./65 ± 5% RH | 97.1 | 1.0 | 1.9 |
| | | EDTA, | 1M | 5 ± 3° C. | 98.2 | 0.8 | 1.0 |
| | | 20 mg/mL | | 25 ± 2° C./60 ± 5% RH | 97.0 | 0.9 | 2.2 |
| | | Propylene | | 30 ± 2° C./65 ± 5% RH | 96.4 | 0.9 | 2.7 |
| | | Glycol, | 2M | 5 ± 3° C. | 98.2 | 1.0 | 0.8 |
| | | pH 8.3 with | | 25 ± 2° C./60 ± 5% RH | 95.0 | 1.0 | 4.0 |
| | | fibril seeds | | 30 ± 2° C./65 ± 5% RH | 93.3 | 1.4 | 5.3 |
| | | | 3M | 5 ± 3° C. | 96.6 | 1.1 | 2.3 |
| | | | | 25 ± 2° C./60 ± 5% RH | 94.9 | 0.7 | 4.4 |
| | | | 6M | 5 ± 3° C. | 96.6 | 0.7 | 2.7 |
| | | | | 25 ± 2° C./60 ± 5% RH | 87.5 | 1.8 | 10.6 |

### Effect of Antioxidants on Stability of Compound 1

### Introduction

The degradation studies have shown that Compound 1 may be susceptible to oxidation. The inclusion of EDTA, a commonly used antioxidant, is effective in mitigating oxidation in the solution state. Other excipients may also be considered to curtail oxidation. In the current study, EDTA, citrate and methionine were evaluated as antioxidants for Compound 1 solution formulations. A sample without any antioxidant was included as a control.

### Materials and Methods

Compound 1 API and excipients used in the study are detailed in Table 14. All other laboratory reagents were used as is.

TABLE 14
Study materials

| Material | ID | Expiration |
|---|---|---|
| Compound I | BO1706P002 | n/a |
| (Peptide content = 83.7%) | | |
| (Purity = 97.1%) | | |
| Tris | D313752 | July, 2023 |
| MW = 121.136 g/mol | | |
| Glycerin, synthetic | D097358 | October, 2022 |
| Citric acid, anhydrous | D251666 | May, 2023 |
| MW = 192.123 g/mol | | |
| Methionine | SLCD9864 | n/a |
| MW = 149.21 g/mol | | |
| EDTA disodium salt | CDBB4550V | n/a |
| dihydrate | | |
| MW = 372.24 g/mol | | |
| NaOH, 1N ^{b} | 164777 | July, 2022 |
| Purified water | AE29421220 | July, 2022 |

^{a} The amount of Compound 1 is adjusted to account for the quantity by HPLC reported in the Certificate of Analysis.
^{b} Quantity sufficient to adjust pH

The formulations comprising Compound 1 to be studied are provided in Table 15.

TABLE 15
Compound I formulation composition

| | Compound I Drug Product matrix | | | | |
|---|---|---|---|---|---|
| | | | Tonicity | | |
| | Active | Buffer | agent | | |
| | Compound | Tris | Glycerin, | | |
| Lot | 1 | (mg/mL, | synthetic | | |
| No. | (mg/mL) | 10 mM) | (mg/mL) | Antioxidant | pH |
| 25-1 | 20 | 1.21 | 20 | — | 8.2 |
| 25-2 | 20 | 1.21 | 20 | EDTA | 8.2 |
| | | | | 0.5 mg/mL | |
| 25-3 | 20 | 1.21 | 20 | Citrate | 8.2 |
| | | | | 1.92 mg/mL (10 mM) | |
| 25-4 | 20 | 1.21 | 20 | Methionine | 8.2 |
| | | | | 1.49 mg/mL (10 mM) | |
| 26 | 20 | 1.21 | 20 | Methionine | 8.2 |
| | | | | 14.9 mg/mL (100 mM) | |

Solutions were filtered through 0.22 mm PVDF filters. In a laminar flow hood, the solutions were filled into glass vials. Vials were capped, and stored at 5° C., 25° C. and 30° C. Four formulations comprising Compound 1 were prepared to evaluate the stabilization efficacy of antioxidants, namely, EDTA, citrate and methionine. A control sample that does not contain any antioxidant was also included (Table 15, Lot Nos. 25-1, 25-2, 25-3 and 25-4). Subsequently, a fifth sample containing higher concentration of methionine (100 mM vs. 10 mM) was prepared to evaluate the effect of methionine concentration (Table 15, Lot No. 26). Samples were prepared, and stored at 5° C., 25° C. and 30° C. for up to 3 months. At appropriate times, stability-indicating assays were performed to assess the physical and chemical stability.

### Appearance of Compound 1 Formulations

Appearance of the Compound 1 formulations after three months by visual inspection showed distinct differences among the formulations (Table 15). At 5° C., solutions were colorless. However, at 25° C. and 30° C., the control and the methionine-containing samples were slightly yellow, while the EDTA and citrate-containing samples remained colorless. Color change is often an indication of chemical degradation. At 5° C., rate of chemical degradation may be sufficiently slow regardless of stabilizers, and hence, all solutions appear colorless. At elevated temperatures, the diverging degradation kinetics reflect the effect of antioxidants.

TABLE 16
Appearance of Compound 1 formulations after three (3) months

| | | Temperature | |
|---|---|---|---|
| Lot No. | Stabilizer | (° C.) | Appearance |
| 25-1 | None; Control | 5 | Colorless |
| 25-2 | EDTA, 0.5 mg/mL | | Colorless |
| 25-3 | Citrate, 10 mM | | Colorless |
| 25-4 | Methionine, 10 mM | | Not available |
| 25-1 | None; Control | 25 | Slightly yellow |
| 25-2 | EDTA, 0.5 mg/mL | | Colorless |
| 25-3 | Citrate, 10 mM | | Colorless |
| 25-4 | Methionine, 10 mM | | Slightly yellow |
| 25-1 | None; Control | 30 | Slightly yellow |
| 25-2 | EDTA, 0.5 mg/mL | | Colorless |
| 25-3 | Citrate, 10 mM | | Colorless |
| 25-4 | Methionine, 10 mM | | Slightly yellow |

### Effect of Antioxidant on the Chemical Stability of Compound 1 by RP-HPLC

The data in Table 17 shows that chemical degradation of Compound 1 was not substantial at the refrigerated condition. At 25° C. and 30° C., there are significant differences in stabilization efficacy. Without any antioxidant, the control sample showed rapid chemical degradation. Methionine, a commonly used antioxidant in monoclonal antibody formulations, demonstrated lower stabilization efficacy (either at 10 mM or 100 mM) in comparison to EDTA and citrate. Chemical degradation was suppressed by either EDTA or citrate, with EDTA being slightly more effective than citrate.

TABLE 17
Compound 1 total impurities by RP-HPLC

| | | Total Impurities (%) | | | | |
|---|---|---|---|---|---|---|
| | | | | | 25-4 | 26 |
| Temp | Time | 25-1 | 25-2 | 25-3 | (10 mM | (100 mM |
| (° C.) | (month) | (Control) | (EDTA) | (Citrate) | methionine) | methionine |
| T = 5° C. | 0 | 2.5 | 2.4 | 2.5 | 2.5 | 2.8 |
| | 3 | 3.1 | 2.7 | 2.9 | 3.1 | 3.0 |
| T = 25° C. | 0 | 2.5 | 2.4 | 2.5 | 2.5 | 2.8 |
| | 1 | 6.4 | 4.5 | 4.9 | 6.0 | 5.9 |
| | 3 | 13.6 | 8.5 | 9.4 | 12.9 | — |
| T = 30° C. | 0 | 2.5 | 2.4 | 2.5 | 2.5 | 2.8 |
| | 1 | 8.3 | 5.5 | 6.0 | 7.5 | 7.5 |
| | 3 | 19.8 | 11.6 | 14.0 | 17.6 | — |

Data from experiments described hereinabove demonstrate that oxidation of Trp is one of the major degradation pathways. The data in Table 18 shows the possible Trp oxidation was almost completely suppressed by EDTA or citrate, with a lesser effect obtainable from methionine.

TABLE 18
Compound 1 impurities related to the oxidation of Trp by RP-HPLC

| | | Trp oxidation (%) | | | | |
|---|---|---|---|---|---|---|
| | | | | | 25-4 | 26 |
| Temp | Time | 25-1 | 25-2 | 25-3 | (10 mM | (100 mM |
| (° C.) | (month) | (Control) | (EDTA) | (Citrate) | methionine) | methionine |
| T = 5° C. | 0 | 0.1 | 0.1 | 0.1 | 0.1 | 0.1 |
| | 3 | 0.1 | 0.1 | 0.0 | 0.2 | 0.1 |
| T = 25° C. | 0 | 0.1 | 0.1 | 0.1 | 0.1 | 0.1 |
| | 1 | 0.4 | 0.1 | 0.1 | 0.3 | 0.3 |
| | 3 | 1.0 | 0.1 | 0.1 | 0.9 | — |
| T = 30° C. | 0 | 0.1 | 0.1 | 0.1 | 0.1 | 0.1 |
| | 1 | 0.5 | 0.1 | 0.1 | 0.4 | 0.4 |
| | 3 | 1.3 | 0.2 | 0.2 | 1.0 | — |

### Effect of Antioxidant on the Physical Stability of Compound 1

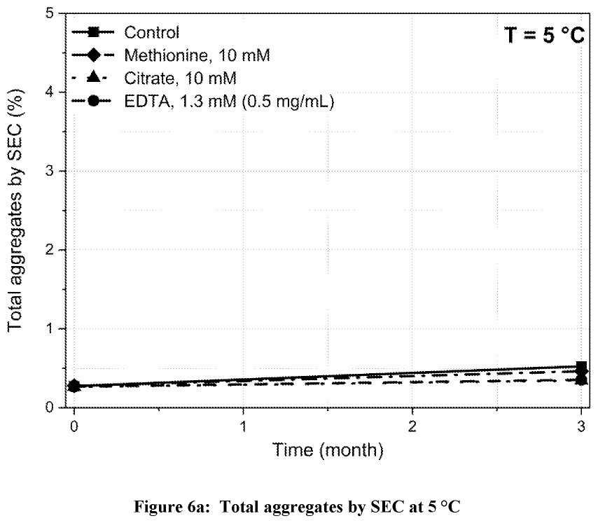

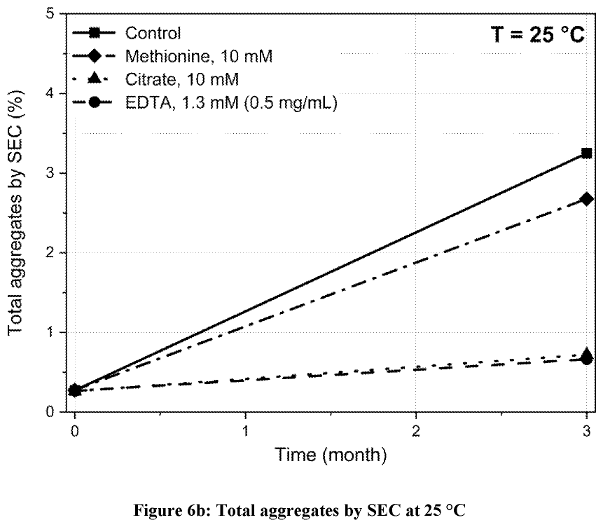

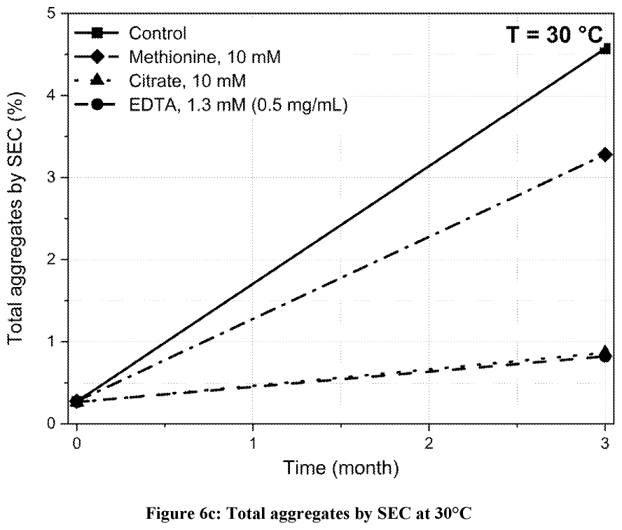

The formation of covalent aggregates was evaluated by size-exclusion chromatography (SEC). The total aggregates as measured by SEC over three months is shown in FIG. 6a for vials stored at 5° C., in FIG. 6b for vials stored at 25° C. and in FIG. 6c for vials stored at 30° C. All formulations were well behaved at 5° C. At 25° C. and 30° C., EDTA and citrate demonstrated significant stabilization efficacy.

In addition to SEC, the three-month samples were tested for subvisible particulate matter using light obscuration (HIAC) and flow imaging (MFI) methods. The experimental data is shown in Table 19. No obvious trends are observed. Particle counts by HIAC are all within specifications for the ≥10 μm and ≥25 μm measurements.

TABLE 19
Compound 1 total aggregates by SEC

| | | Total Aggregates (%) | | | |
|---|---|---|---|---|---|
| | | | | | 25-4 |
| | Time | 25-1 | 25-2 | 25-3 | (10 mM |
| Temp (° C.) | (month) | (Control) | (EDTA) | (Citrate) | methionine) |
| T = 5° C. | 0 | 0.3 | 0.3 | 0.3 | 0.3 |
| | 3 | 0.5 | 0.4 | 0.3 | 0.5 |
| T = 25° C. | 0 | 0.3 | 0.3 | 0.3 | 0.3 |
| | 3 | 3.2 | 0.7 | 0.7 | 2.7 |
| T = 30° C. | 0 | 0.3 | 0.3 | 0.3 | 0.3 |
| | 3 | 4.6 | 0.8 | 0.9 | 3.3 |

1. A pharmaceutical formulation comprising:
(i) a compound of the following formula
His-Xaa2-Gln-Gly-Thr-Phe-Thr-Ser-Asp-Tyr-Ser-Lys-Tyr-Leu-Asp-Glu-Lys-Lys-Ala-Lys-Glu-Phe-Val-Glu-Trp-Leu-Leu-Xaa28-Gly-Gly-Pro-Ser-Ser-Gly
wherein
Xaa2 is Aib;
Xaa28 is Glu or Ser;
Lys at position 20 is chemically modified by conjugation of the epsilon-amino group of the Lys side chain with a C14-C24 fatty acid via a linker between the Lys at position 20 and the C14-C24 fatty acid, wherein the linker is ([2-(2-aminoethoxy)-ethoxy]-acetyl)2-(γ-Glu)t, wherein t is 1 or 2; and
the C-terminal amino acid is optionally amidated (SEQ ID NO: 5);
(ii) a buffer;
(iii) a tonicity agent; and
(iii) an antioxidant,
wherein the pH of the formulation is 7.8-9.0.

2. A pharmaceutical formulation according to claim 1, wherein the compound is selected from the group consisting of:
(a) a compound of the following formula:
His-Xaa2-Gln-Gly-Thr-Phe-Thr-Ser-Asp-Tyr-Ser-Lys-Tyr-Leu-Asp-Glu-Lys-Lys-Ala-Lys-Glu-Phe-Val-Glu-Trp-Leu-Leu-Glu-Gly-Gly-Pro-Ser-Ser-Gly
wherein Xaa 2 is Aib;
Lys at position 20 is chemically modified by conjugation of the epsilon-amino group of the Lys side chain with ([2-(2-aminoethoxy)-ethoxy]-acetyl)2-(γ-Glu)-CO—(CH2)18CO2H; and
the C-terminal amino acid is amidated (SEQ ID NO: 1);
(b) a compound of the following formula:
His-Xaa2-Gln-Gly-Thr-Phe-Thr-Ser-Asp-Tyr-Ser-Lys-Tyr-Leu-Asp-Glu-Lys-Lys-Ala-Lys-Glu-Phe-Val-Glu-Trp-Leu-Leu-Ser-Gly-Gly-Pro-Ser-Ser-Gly
wherein Xaa 2 is Aib;
Lys at position 20 is chemically modified by conjugation of the epsilon-amino group of the Lys side chain with ([2-(2-aminoethoxy)-ethoxy]-acetyl)2-(γ-Glu)₂-CO—(CH2)18CO2H; and
the C-terminal amino acid is amidated (SEQ ID NO: 2);
(c) a compound of the following formula:
His-Xaa2-Gln-Gly-Thr-Phe-Thr-Ser-Asp-Tyr-Ser-Lys-Tyr-Leu-Asp-Glu-Lys-Lys-Ala-Lys-Glu-Phe-Val-Glu-Trp-Leu-Leu-Glu-Gly-Gly-Pro-Ser-Ser-Gly
wherein Xaa 2 is Aib;
Lys at position 20 is chemically modified by conjugation of the epsilon-amino group of the Lys side chain with ([2-(2-aminoethoxy)-ethoxy]-acetyl)2-(γ-Glu)-CO—(CH2)16CO2H; and
the C-terminal amino acid is amidated (SEQ ID NO: 3);
(d) a compound of the following formula:
His-Xaa2-Gln-Gly-Thr-Phe-Thr-Ser-Asp-Tyr-Ser-Lys-Tyr-Leu-Asp-Glu-Lys-Lys-Ala-Lys-Glu-Phe-Val-Glu-Trp-Leu-Leu-Ser-Gly-Gly-Pro-Ser-Ser-Gly
wherein Xaa 2 is Aib;
Lys at position 20 is chemically modified by conjugation of the epsilon-amino group of the Lys side chain with ([2-(2-aminoethoxy)-ethoxy]-acetyl)2-(γ-Glu)₂-CO—(CH2)16CO2H; and
the C-terminal amino acid is amidated (SEQ ID NO: 4).

3. A pharmaceutical formulation according to claim 1,
wherein the compound has the following formula:
His-Xaa2-Gln-Gly-Thr-Phe-Thr-Ser-Asp-Tyr-Ser-Lys-Tyr-Leu-Asp-Glu-Lys-Lys-Ala-Lys-Glu-Phe-Val-Glu-Trp-Leu-Leu-Glu-Gly-Gly-Pro-Ser-Ser-Gly
wherein Xaa 2 is Aib;
Lys at position 20 is chemically modified by conjugation of the epsilon-amino group of the Lys side chain with ([2-(2-aminoethoxy)-ethoxy]-acetyl)2-(γ-Glu)-CO—(CH2)18CO2H; and
the C-terminal amino acid is amidated (SEQ ID NO: 1).

4. A pharmaceutical formulation according to claim 1, wherein the formulation comprises 1 mg/mL to 100 mg/mL of the compound.

5. A pharmaceutical formulation according to claim 1, wherein the buffer is selected from the group consisting of a phosphate buffer and a tris(hydroxymethyl)aminomethane (or 2-amino-2-hydroxymethyl-propane-1,3-diol[(HOCH₂)₃CNH₂]) buffer.

6. A pharmaceutical formulation according to claim 1, wherein the formulation comprises 1 mM to 20 mM of buffer.

7. A pharmaceutical formulation according to claim 5, wherein the buffer is a tris(hydroxymethyl)aminomethane (Tris) buffer.

8. A pharmaceutical formulation according to claim 7, wherein the formulation comprises 10 mM Tris buffer.

9. A pharmaceutical formulation according to claim 1, wherein the tonicity agent is selected from the group consisting of mannitol, sucrose, trehalose, propylene glycol, glycerin, sodium chloride and arginine hydrochloride.

10. A pharmaceutical formulation according to claim 1, wherein the formulation comprises 5 mg/mL to 150 mg/mL of the tonicity agent.

11. A pharmaceutical formulation according to claim 9, wherein the tonicity agent is mannitol.

12. A pharmaceutical formulation according to claim 11, wherein the formulation comprises 45-55 mg/mL of mannitol.

13. A pharmaceutical formulation according to claim 1, wherein the antioxidant is selected from the group consisting of radical scavengers, chelators or chain terminators.

14. A pharmaceutical formulation according to claim 1, wherein the formulation comprises 0.05-10.0 mg/mL of the antioxidant.

15. A pharmaceutical formulation according to claim 1, wherein the antioxidant is selected from the group consisting of EDTA, citric acid, ascorbic acid, butylated hydroxytoluene (BHT), butylated hydroxy anisole (BHA), sodium sulfite, p-amino benzoic acid, glutathione, propyl gallate, histidine, cysteine, methionine, ethanol and N-acetyl cysteine.

16. A pharmaceutical formulation according to claim 15, wherein the antioxidant is EDTA.

17. A pharmaceutical formulation according to claim 16, wherein the formulation comprises 0.2-1.0 mg/mL of EDTA.

18. A pharmaceutical formulation according to claim 17, wherein the formulation comprises 0.5 mg/mL of EDTA.

19. A pharmaceutical formulation according to claim 15, wherein the antioxidant is citric acid.

20. A pharmaceutical formulation according to claim 19, wherein the formulation comprises 5 mM to 15 mM citric acid.

21. A pharmaceutical formulation according to claim 20, wherein the formulation comprises 8 mM to 12 mM citric acid.

22. A pharmaceutical formulation according to claim 21, wherein the formulation comprises 10 mM citric acid.

23. A pharmaceutical formulation according to claim 1, wherein the pH of the formulation is 8.0-8.6.

24. A pharmaceutical formulation according to claim 23, wherein the pH of the formulation is 8.0-8.3.

25. A pharmaceutical formulation according to claim 1, comprising:
(i) 1 mg/mL to 100 mg/mL of the compound of the following formula:
His-Xaa2-Gln-Gly-Thr-Phe-Thr-Ser-Asp-Tyr-Ser-Lys-Tyr-Leu-Asp-Glu-Lys-Lys-Ala-Lys-Glu-Phe-Val-Glu-Trp-Leu-Leu-Glu-Gly-Gly-Pro-Ser-Ser-Gly
wherein Xaa 2 is Aib;
Lys at position 20 is chemically modified by conjugation of the epsilon-amino group of the Lys side chain with ([2-(2-aminoethoxy)-ethoxy]-acetyl)2-(γ-Glu)-CO—(CH2)18CO2H; and
the C-terminal amino acid is amidated (SEQ ID NO: 1);
(ii) 10 mM of Tris buffer;
(iii) 46 mg/mL of mannitol;
(iv) 0.5 mg/mL of EDTA,
wherein the pH of the formulation is 8.0-8.3.

26. A method of treating and/or preventing type 2 diabetes, wherein the method comprises administering to a patient a therapeutically effective amount of a pharmaceutical formulation of claim 1.

27. A method of treating and/or preventing obesity, wherein the method comprises administering to a patient a therapeutically effective amount of a pharmaceutical formulation of claim 1.

28. A method of treating and/or preventing nonalcoholic fatty liver disease (NAFLD), wherein the method comprises administering to a patient a therapeutically effective amount of a pharmaceutical formulation of claim 1.

29. A method of treating and/or preventing nonalcoholic steatohepatitis (NASH), wherein the method comprises administering to a patient a therapeutically effective amount of a pharmaceutical formulation of claim 1.

30-37. (canceled)
